(19) United States
(12) Patent Application Publication
(10) Pub. No.: US 2026/0103701 A1
(43) Pub. Date: Apr. 16, 2026

(54) TET-ASSISTED PYRIDINE BORANE SEQUENCING

(71) Applicant: Exact Sciences Innovation LTD., Oxford (GB)

(72) Inventors: Bronwen Miller, Kidlington (GB); Rosemary Wilson, Oxfordshire (GB); Luca Tosti, Oxford (GB); Abram Vaccaro, Rio, WI (US); Chunxiao Song, Oxford (GB); Sarah Walsh, Oxford (GB)

(21) Appl. No.: 19/115,509

(22) PCT Filed: Oct. 3, 2023
(86) PCT No.: PCT/US23/75823

§ 371 Date: Mar. 26, 2025

Related U.S. Application Data
(60) Provisional application No. 63/413,069, filed on Oct. 4, 2022.
(60) Provisional application No. 63/460,364, filed on Apr. 19, 2023.

(51) Int. Cl. C12N 15/10; C12Q 1/6806; C12Q 1/6874

(52) U.S. Cl. CPC C12N 15/1065; C12N 15/1068; C12Q 1/6806; C12Q 1/6874

(57) ABSTRACT

Methods of amplifying libraries after introduction of dihydrouracil (DHU) residues by methods such as TET-assisted Pyridine Borane Sequencing (TAPS), and variants of TAPS including TAPS with blocking by β-glucosylation (TAPSβ) and Chemically-assisted Pyridine Borane Sequencing (CAPS) are described. The methods comprise introducing DHU residues into a nucleic acid sample and preparation of a sequencing library by a complementary strand synthesis step reaction with a first polymerase or polymerase mixture that is tolerant of DHU residues and/or products resulting from the introduction of the DHU residues and/or the TAPS process followed by exponential amplification. Improved methods for conversion of oxidized nucleotide residues to DHU are also described.

### FIELD

The present disclosure provides compositions and methods related to TET-assisted Pyridine Borane Sequencing (TAPS). In particular, the present disclosure provides optimized methods for generating and sequencing TAPS libraries.

### BACKGROUND

5-Methylcytosine (5mC) and 5-hydroxymethylcytosine (5hmC) are the two major epigenetic marks found in the mammalian genome. 5hmC is generated from 5mC by the ten-eleven translocation (TET) family dioxygenases. TET can further oxidize 5hmC to 5-formylcytosine (5fC) and 5-carboxylcytosine (5 caC), which exists in much lower abundance in the mammalian genome compared to 5mC and 5hmC (10-fold to 100-fold lower than that of 5hmC). Together, 5mC and 5hmC play crucial roles in a broad range of biological processes from gene regulation to normal development. Aberrant DNA methylation and hydroxymethylation have been associated with various diseases and are well-accepted hallmarks of cancer. Therefore, the determination of 5mC and 5hmC in DNA sequence is not only important for basic research, but also is valuable for clinical applications, including diagnosis and therapy.

The current gold standard and most widely used method for DNA methylation and hydroxymethylation analysis is bisulfite sequencing (BS), and its derived methods such as TET-assisted bisulfite sequencing (TAB-Seq) and oxidative bisulfite sequencing (oxBS). Likewise, bisulfite sequencing is the most well-established method for assaying whole genome DNA methylation. All of these methods employ bisulfite treatment to convert unmethylated cytosine to uracil while leaving 5mC and/or 5hmC intact. Through PCR amplification of the bisulfite-treated DNA, which reads uracil as thymine, the modification information of each cytosine can be inferred at a single base resolution (where the transition of C to T provides the location of the unmethylated cytosine). There are, however, at least two main drawbacks to bisulfite sequencing. First, bisulfite treatment is a harsh chemical reaction, which degrades more than 90% of the DNA due to depurination under the required acidic and thermal conditions. This degradation severely limits its application to low-input samples, such as clinical samples including circulating cell-free DNA and single-cell sequencing. Second, bisulfite sequencing relies on the complete conversion of unmodified cytosine to thymine. Unmodified cytosine accounts for approximately 95% of the total cytosine in the human genome. Converting all these positions to thymine severely reduces sequence complexity, leading to poor sequencing quality, low mapping rates, uneven genome coverage and increased sequencing cost, as well as reducing the ability to call variants. Bisulfite sequencing methods are also susceptible to false detection of 5mC and 5hmC due to incomplete conversion of unmodified cytosine to thymine.

Sequencing of DNA samples which have been treated to modify the naturally occurring bases can be difficult, especially when using massively parallel next generation sequencing (NGS) methods. In particular, problems occur with underrepresentation of highly methylated regions of interest. The present invention provides a solution to this problem.

### SUMMARY

Embodiments of the present disclosure include methods of sequencing libraries after introduction of dihydrouracil (DHU) residues by methods such as TET-assisted Pyridine Borane Sequencing (TAPS), and variants of TAPS including TAPS with blocking by β-glucosylation (TAPSβ) and Chemically-assisted Pyridine Borane Sequencing (CAPS). In accordance with these embodiments, the method includes introducing DHU residues into a nucleic acid sample and preparation of a sequencing library by a synthesis step with a first polymerase or polymerase mixture that is tolerant of DHU residues and/or products resulting from the introduction of the DHU residues and/or the TAPS process followed by exponential amplification.

Accordingly, in some embodiments, the present invention provides a method for amplifying a target nucleic acid molecule comprising dihydrouracil (DHU) residues comprising: synthesizing one or more complementary strands of the target nucleic acid comprising DHU residues with a first polymerase or polymerase mixture that is tolerant of DHU residues and/or products resulting from the introduction of the DHU residues and/or the TAPS process to provide a target nucleic acid mixture comprising the target nucleic acid comprising DHU residues and one or more complementary strands; and exponentially amplifying the target nucleic acid mixture to provide amplified target nucleic acid.

In some embodiments, the first polymerase or polymerase mixture has an error rate of greater than 5.0×10⁻⁵. In some embodiments, the first polymerase or polymerase mixture is selected from the group consisting of Bst3.0 polymerase, Sulpholobus polymerase IV, a combination of Bst3.0 polymerase and Sulpholobus polymerase IV, Klenow polymerase, Klenow exo-polymerase, Polk polymerase, Mu-mLV reverse transcriptase, SD polymerase, Tth polymerase, OneTaq polymerase, a combination of OneTaq and Tth polymerase, 5D4 polymerase, a 5D4 polymerase blend with Taq polymerase, and SD polymerase.

In some embodiments, the first polymerase is thermolabile. In some embodiments, the first polymerase is thermostable. In some embodiments, the step of exponentially amplifying the complementary strand of the target nucleic acid utilizes the first polymerase or polymerase mixture that is tolerant of DHU residues and/or products resulting from the introduction of the DHU residues and/or the TAPS process.

In some embodiments, the step of exponentially amplifying the pre-amplified target nucleic acid utilizes a second polymerase or polymerase. In some embodiments, the second polymerase or polymerase has an error rate of less than 5.0×10⁵. In some embodiments, the second polymerase or polymerase mixture has an error rate of less than 1.0×10⁻⁶. In some embodiments, the second polymerase is selected from the group consisting of GoTaq polymerase and KAPA HiFi Uracil+ polymerase. In some embodiments, the polymerase having an error rate of less than 5.0×10⁻⁵ is thermostable.

In some embodiments, the first polymerase and the second polymerase are provided in a mastermix.

In some embodiments, synthesizing a complementary strand of the target nucleic acid comprising DHU residues with a first polymerase or polymerase mixture further comprises synthesis in a buffer comprising from about 0.5-0.75 mM MnSO₄.

In some embodiments, the methods further comprise quantifying the amplified target nucleic acid.

In some embodiments, the methods further comprise the step of sequencing the exponentially amplified target nucleic acid.

In some embodiments, the target nucleic acid comprising DHU residues has sequencing library adapters ligated to each end. In some embodiments, the sequencing library adapters comprise an index sequence. In some embodiments, the sequencing library adapters comprise sequences complementary to sequencing primers. In some embodiments, the sequencing library adapters comprise sequences complementary to indexing primers.

In some embodiments, the step of synthesizing a complementary strand of the target nucleic acid comprising DHU residues further comprises annealing forward and/or reverse primer(s) to the sequencing library adapters.

In some embodiments, the step of exponentially amplifying the complementary strand of the target nucleic acid comprises annealing library amplification primers to the pre-amplified target nucleic acid.

In some embodiments, the sequencing is performed by massively parallel sequencing.

In some preferred embodiments, the step of contacting the oxidized nucleic acid sample comprising 5 caC and/or 5fC with a borane reducing agent further comprises reacting the oxidized nucleic acid sample with the borane reducing agent in a reaction mixture comprising from 45.0% to 52.5% DMSO by volume.

In some preferred embodiments, the step of contacting the oxidized nucleic acid sample comprising 5 caC and/or 5fC with a borane reducing agent further comprises reacting the oxidized nucleic acid sample with the borane reducing agent at a temperature of from 45.0 degrees Celsius to 52.5 degrees Celsius.

In some preferred embodiments, the step of contacting the oxidized nucleic acid sample comprising 5 caC and/or 5fC with a borane reducing agent further comprises reacting the oxidized nucleic acid sample with the borane reducing agent for a period of time from 45 to 60 minutes.

In some preferred embodiments, the present invention further provides methods for converting 5-carboxylcytosine (5 caC) and/or 5-formylcytosine (5fC) to dihydrouracil (DHU) comprising contacting a nucleic acid sample comprising 5 caC and/or 5fC with a borane reducing agent in a reaction mixture comprising from 45.0% to 52.5% DMSO by volume. In some preferred embodiments, the methods further comprise reacting the oxidized nucleic acid sample with the borane reducing agent at a temperature of from 45.0 degrees Celsius to 52.5 degrees Celsius. In some preferred embodiments, the methods further comprise reacting the oxidized nucleic acid sample with the borane reducing agent for a period of time from 45 to 60 minutes.

In some preferred embodiments, the borane reducing agent comprises an agent selected from the group consisting of 2-picoline borane (pic-BH3), borane, sodium borohydride, sodium cyanoborohydride, and sodium triacetoxyborohydride. In some preferred embodiments, the borane reducing agent comprises sodium borohydride. In some preferred embodiments, the borane reducing agent comprises sodium cyanoborohydride. In some preferred embodiments, the borane reducing agent comprises sodium triacetoxyborohydride. In some preferred embodiments, the borane reducing agent comprises 2-picoline borane.

In some preferred embodiments, the methods comprise contacting the nucleic acid sample with an oxidizing agent prior to contacting with a borane reducing agent. In some preferred embodiments, the oxidizing agent is a ten-eleven translocation (TET) enzyme. In some preferred embodiments, the TET enzyme comprises human TET1, human TET2, human TET3, murine TET1, murine TET2, murine TET3, Naegleria TET (NgTET), Coprinopsis cinerea (CcTET), or derivatives or analogues thereof. In some preferred embodiments, the oxidizing agent comprises a chemical oxidizing agent. In some preferred embodiments, the chemical oxidizing agent comprises manganese oxide (MnO₂), potassium ruthenate (K₂RuO₄), potassium perruthenate (KRuO₄) or Cu(II)/TEMPO.

In some preferred embodiments, the methods further comprise adding a blocking group to one or more modified cytosines in the nucleic acid sample.

In some preferred embodiments, the methods further comprise sequencing the nucleic acid sample after contacting with the borane reducing agent to identify converted cytosine bases.

### BRIEF DESCRIPTION OF THE DRAWINGS

FIG. 1. Normalized GC bias from NGS sequencing for fully methylated lambda spike in from two TAPS treated samples compared to two samples not treated with TAPS.

FIG. 2. Schematic of complementary strand synthesis steps prior to amplification.

FIG. 3. Normalized GC bias from NGS sequencing for fully methylated lambda spike in with a Bst 3.0 complementary strand synthesis step prior to amplification with or without the denaturation step.

FIG. 4. Normalized GC bias from NGS sequencing for fully methylated lambda spike in with a Bst 3.0 complementary strand synthesis step prior to amplification with separate buffer or spike in option.

FIG. 5. Normalized GC bias from NGS sequencing for fully methylated lambda spike in with a Bst 3.0+/−Sulpholobus pol IV complementary strand synthesis step prior to amplification.

FIG. 6. Normalized GC bias from NGS sequencing for fully methylated lambda spike in with a Bst 3.0+/−WarmStart RTx complementary strand synthesis step prior to amplification.

FIG. 7. Normalized GC bias from NGS sequencing for fully methylated lambda spike in with a Bst 3.0 or M-MuLV RT complementary strand synthesis step prior to amplification.

FIG. 8. Normalized GC bias from NGS sequencing for fully methylated lambda spike in with a OneTaq™ and Tth complementary strand synthesis step prior to amplification.

FIG. 9. Normalized GC bias from NGS sequencing for fully methylated lambda spike in with a OneTaq™ and Tth complementary strand synthesis step prior to amplification, or only OneTaq™, or only Tth.

FIG. 10. Normalized GC bias from NGS sequencing for fully methylated lambda spike in with a OneTaq™ and Tth complementary strand synthesis step prior to amplification, or only Taq, both with 0.75 mM or 0 mM MnSO₄.

FIG. 11. Normalized GC bias from NGS sequencing for fully methylated lambda spike in with a Polymerase κ (kappa) complementary strand synthesis step prior to amplification.

FIG. 12. Normalized GC bias from NGS sequencing for fully methylated lambda spike in with a DNA Pol I Klenow fragment exo-complementary strand synthesis step prior to amplification.

FIG. 13. Normalized GC bias from NGS sequencing for fully methylated lambda spike in with a SD polymerase complementary strand synthesis step prior to amplification with or without denaturation.

FIG. 14. Normalized GC bias from NGS sequencing for fully methylated lambda spike in with a 5D4 complementary strand synthesis step prior to 1 amplification, either alone or as a spike in option.

FIG. 15. Normalized GC bias from NGS sequencing for fully methylated lambda spike in with library amplification with Kapa Hifi Uracil+ as standard, or with 5D4 as a spike in option, or replacing Hifi U+ with 10:1 Taq: 5D4.

FIG. 16A-16B. Average modification rate (16A) and depth (16B) for selected marker regions from high coverage whole genome sequencing of NA12878. Conditions shown are no complementary strand synthesis step prior to amplification (control, red), and a Bst3.0 synthesis step prior to amplification with (98, green) and without (no98, yellow) initial 1 min 98° C. denaturation step.

FIG. 17A-17B. Average modification rate (17A) and normalized depth (17B, average depth shown as dashed line) for selected marker regions from high coverage whole genome sequencing of NA12878. Conditions shown are no complementary strand synthesis step prior to amplification (KU_std, green), a Bst3.0 complementary strand synthesis step prior to amplification as spike into Kapa Hifi Uracil+ with added 0.75 mM MnSO (4) (Bst_spike_Mn, yellow) and a OneTaq and Tth complementary strand synthesis step prior to amplification (CS_std, red).

FIG. 18A-18B. Average modification rate (18A) and normalized depth (18B) for selected marker regions from Whole Genome Sequencing (WGS) sequencing of pooled normal cfDNA. Conditions shown are a Bst3.0 complementary strand synthesis step prior to amplification as spike into Kapa Hifi Uracil+ (Bst), and a SD polymerase complementary strand synthesis step prior to amplification (SD).

FIG. 19A-19B. Average modification rate (19A) and normalized depth (19B) for selected marker regions from Whole Genome Sequencing (WGS) sequencing of pooled normal cfDNA. Conditions shown are no pre-extension (KU), a Bst3.0 complementary strand synthesis step prior to amplification as spike into Kapa Hifi Uracil+ (Bst) and a OneTaq™ and Tth complementary strand synthesis step prior to amplification (OTT).

FIG. 20A-20B. Average modification rate (left) and normalized depth (right) for selected marker regions from hybridization capture targeted sequencing of pooled normal cfDNA. Conditions shown are no pre-extension (KU), a Bst3.0 complementary strand synthesis step prior to amplification as spike into Kapa Hifi Uracil+ (Bst) and a OneTaq and Tth complementary strand synthesis step prior to amplification (OTT).

FIG. 21. Normalized GC bias from NGS sequencing for fully methylated lambda spike in with a Bst 3.0 complementary strand synthesis step prior to amplification with separate buffer or spike in option using Swift BioScience Accel Methyl-Seq kit.

FIG. 22. Normalized GC bias from NGS sequencing for fully methylated lambda spike in with a Bst 3.0 or DNA pol I Klenow Fragment exo-complementary strand synthesis step prior to amplification using Claret Bioscience SRSLY kit.

FIG. 23. Normalized GC bias from NGS sequencing for fully methylated lambda spike in with a Bst 3.0 complementary strand synthesis step prior to amplification using Takara Bio Epi Xplore kit.

FIG. 24. Normalized coverage of selected marker regions with low levels of methylation following TAPS and amplification with different polymerases (Kapa Hifi Uracil+, Bst and OTT).

FIG. 25. Average conversion of selected marker regions with low levels of methylation following TAPS and amplification with different polymerases (Kapa Hifi Uracil+, Bst and OTT).

FIG. 26. Normalized GC bias from NGS sequencing for fully methylated lambda spike in with a SeqAmp polymerase complementary strand synthesis step prior to amplification, a Bst polymerase complementary strand synthesis step prior to amplification, or no separate complementary strand synthesis step prior to amplification.

FIG. 27. Normalized GC bias from NGS sequencing for fully methylated lambda spike in with a Therminator polymerase complementary strand synthesis step prior to amplification, a Bst polymerase complementary strand synthesis step prior to amplification, or no complementary strand separate synthesis step prior to amplification.

### DETAILED DESCRIPTION

Recently, TET-assisted Pyridine Borane Sequencing (TAPS and variants including TAPSB and CAPS), a bisulfite-free DNA methylation sequencing method was developed, as described in PCT/US2019/012627, PCT/IB2020/056435, PCT/IB2021/000630, PCT/IB2021/051091, and PCT/IB2022/000420 each of which is incorporated herein by reference in its entirety. TAPS is based on the use of mild chemistry to detect DNA methylation directly and demonstrated improved sequence quality, mapping rate and coverage compared to bisulfite sequencing, while reducing sequencing cost by half. The combination of direct methylation detection and the non-destructive nature of TAPS makes it useful in a variety of nucleic acid samples including DNA obtained from an organism from the Monera (bacteria), Protista, Fungi, Plantae, and Animalia Kingdoms. The target nucleic acid may also be obtained from a virus. Nucleic acid samples may be obtained from a from a patient or subject, from an environmental sample, or from an organism of interest (e.g., both cellular and circulating cell-free DNA (cfDNA obtained from tissue, a cell, collection of cells, blood, plasma, serum, organ secretion, semen (seminal fluid), vaginal secretions, cerebral spinal fluid (CSF), saliva, mucus, urine, stool, sweat, pancreatic juice, gastric secretions, gastric fluid (gastric lavage), ascitic fluid, synovial fluid, pleural fluid (pleural lavage), pericardial fluid, peritoneal fluid, amniotic fluid, nasal fluid, optic fluid, breast milk, or any other bodily fluid comprising a desired nucleic acid or cfDNA), DNA obtained from biopsies, and DNA obtained from cells, secretions, or tissues from the lymph gland, breast, liver, bile ducts, pancreas, mouth, stomach, colon, rectum, esophagus, small intestine, appendix, duodenum, polyps, gall bladder, anus, prostate, endometrium, vagina, ovary, cervix, skin, bladder, kidney, lung, and/or peritoneum). In other embodiments, the nucleic acid sample may be obtained from a sample that is cancerous, or contains cancerous tissue or cells, or is suspected of being cancerous or suspected of containing cancerous tissue or cells. In some embodiments, the nucleic acid sample is obtained from a subject that has a disease or disorder (e.g., cancer), is suspected of having the disease or disorder, or is being screened to determine the presence of the disease or disorder. In some embodiments, the nucleic acid sample is circulating cell-free DNA (cell-free DNA or cfDNA), for instance DNA found in the blood and is not present within a cell. As would be recognized by one of ordinary skill in the art based on the present disclosure, cfDNA can be isolated from a bodily fluid using methods known in the art. Commercial kits are available for isolation of cfDNA including, for example, the Circulating Nucleic Acid Kit (Qiagen). The nucleic acid sample may result from an enrichment step, including, but is not limited to antibody immunoprecipitation, chromatin immunoprecipitation, restriction enzyme digestion-based enrichment, hybridization-based enrichment, or chemical labeling-based enrichment.

As described further herein, the methods of the present invention provide improved amplification and sequencing of nucleic acid molecules containing DHU residues, preferably DHU residues introduced by a TAPS protocol, or nucleic acid molecules resulting from a TAPS protocol, or nucleic acid molecules containing by-products of a TAPS protocol. Without being limited to any particular theory, it is contemplated that the presence of DHU residues or other by-products introduced by the TAPS protocol cause decreased coverage of methylated regions in target nucleic acids during amplification and sequencing. The present invention addresses this problem. In certain embodiments, a first polymerase or polymerase mixture that is tolerant of DHU residues or other by-products of the TAPS protocol is utilized to produce a complementary strand to a target nucleic acid in at least a first round of amplification followed by an exponential amplification step, optionally with a second polymerase or polymerase mixture. In some preferred embodiments, the first polymerase or polymerase mixture is tolerant of the presence of DHU residues in the target nucleic acid. In some preferred embodiments, the first polymerase or polymerase mixture is tolerant of products resulting from the introduction of the DHU residues into a nucleic acid. In some preferred embodiments, the first polymerase or polymerase mixture is tolerant of products resulting from the TAPS process. In some preferred embodiments, the first polymerase or polymerase mixture is tolerant of the presence of DHU residues in the target nucleic acid and/or is tolerant of products resulting from the introduction of the DHU residues into a nucleic acid and/or is tolerant of products resulting from the TAPS process.

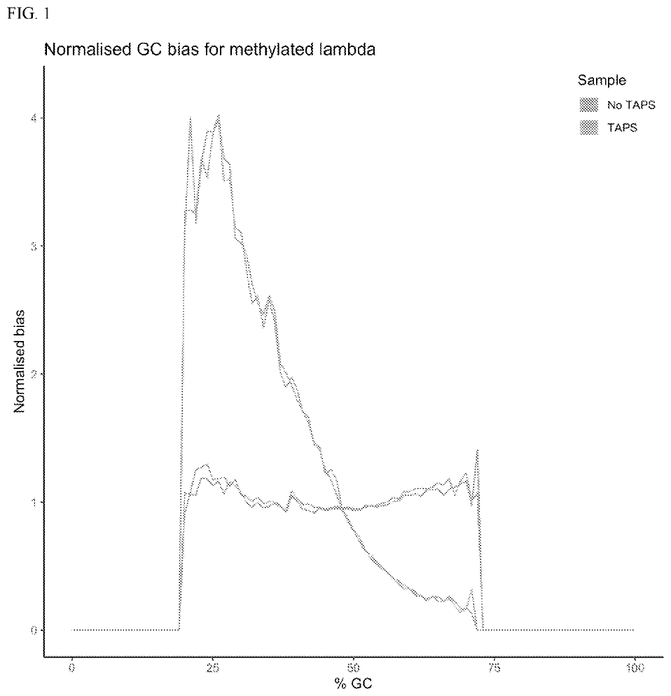

In some preferred embodiments, the first polymerase or polymerase mixture is characterized in having an error rate of greater than 5.0×10⁻⁵ and the second polymerase or polymerase mixture is characterized in having an error rate of less than 5.0×10⁻⁵. In some preferred embodiments, use of the DHU and/or TAPS tolerant polymerase to produce the complementary strand results in improved coverage of methylated (and thus DHU-rich) regions, of a biological target nucleic acid sample that has been processed by a TAPS protocol as compared to the same protocol without the complementary strand synthesis with the DHU and/or TAPS tolerant polymerase. In some embodiments, an improved normalized GC bias for a fully methylated reference or target sequence with complementary strand synthesis with a DHU and/or TAPS tolerant polymerase relative to a control without complementary strand synthesis with a DHU and/or TAPS tolerant polymerase serves as a proxy for demonstrating improved coverage of methylated regions by the DHU and/or TAPS tolerant polymerase. See FIG. 1. It is contemplated that improved GC bias can lead to higher methylation due to less competition between DHU and non-DHU containing strands. In further embodiments, the resulting sequencing libraries are suitable for use in a variety of sequencing methods, include NGS methods.

Results provided herein demonstrate that the methods of the present invention provide improved sequencing coverage of highly methylated regions that are under-represented when standard library preparation and sequencing protocols are utilized. Since the highly methylated regions are of clinical interest, the methods of the present invention are useful, for example, in cancer diagnostics and for the discovery of biomarkers.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide.

As used herein, a “methylated nucleic acid molecule” refers to a nucleic acid molecule that contains one or more methylated nucleotides.

As used herein, a “methylation state”, “methylation profile”, “methylation status,” and “methylation signature” of a nucleic acid molecule refers to the presence of absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

As used herein, “methylation frequency” or “methylation percent (%)” refer to the number of instances in which a molecule or locus is methylated relative to the number of instances the molecule or locus is unmethylated. Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein, the term “error rate” as applied to a polymerase refers to the frequency of errors introduced by a polymerase during replication of a nucleic acid sequence. For example, an error rate 5×10⁻⁵ means that an average of five errors are introduced for every 10⁵ bases replicated.

As used herein, the term “polymerase or polymerase mixture that is tolerant of DHU residues and/or products resulting from the introduction of the DHU residues into the target nucleic acid molecule and/or the TAPS process,” which may be used interchangeably with the term “DHU and/or TAPS tolerant polymerase or polymerase mixture” means a polymerase or polymerase mixture that provides improved coverage of methylated regions of a methylated target DNA sequence that has been treated by a TAPS, TAPSβ, or CAPS protocol as compared to Taq polymerase and/or KAPA HiFi Uracil+ polymerase as assayed by coverage of fully methylated lambda. In some embodiments, assays of GC bias serve as a surrogate for coverage of methylated regions where improved GC bias of one enzyme compared to a reference enzyme (e.g., Taq polymerase or KAPA HiFi Uracil+ polymerase) as determined by amplification and sequencing of a reference sequence (e.g., fully methylated lambda) is indicative of improvement in coverage of methylated regions in a biological sample.

As used herein, the term “improved coverage” in relation to methylated regions in a target sequence refers to [the ability to maintain the proportion of aligned sequence reads corresponding to highly methylated DNA fragments and the proportion of aligned sequence reads corresponding to less highly/non methylated DNA fragments in a more representative fashion, such that the coverage of highly methylated regions is brought closer to the average coverage across the whole genome, and/or the methylation signal is improved.]

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; pinnipeds; and horses.

### 2. TET-ASSISTED PYRIDINE BORANE SEQUENCING (TAPS)

Embodiments of the present disclosure provide a bisulfite-free, base-resolution method for detecting 5-methylcytosine (5mC) and 5-hydroxymethylcytosine (5hmC) in a sequence (e.g., TAPS and associated methods TAPSB and CAPS, referred to collectively as TAPS), including for use with DNA obtained from blood samples (cellular DNA as well as cfDNA) and biopsies. As disclosed in PCT/US2019/012627, U.S. Pat. Publ. 20200370114, U.S. Pat. Publ. 20210317519, PCT/IB2020/056435, PCT/IB2021/000630, PCT/IB2021/051091, and PCT/IB2022/000420, each of which is incorporated herein by reference in its entirety TAPS comprises the use of mild enzymatic and chemical reactions to detect 5mC and 5hmC directly and quantitatively at base-resolution without affecting unmodified cytosine. The present disclosure also provides methods to detect 5-formylcytosine (5fC) and 5-carboxylcytosine (5 caC) at base resolution without affecting unmodified cytosine. Thus, the methods provided herein provide mapping of 5mC, 5hmC, 5fC and 5 caC and overcome the disadvantages of previous methods such as bisulfite sequencing.

In accordance with these embodiments, the methods of the present disclosure include the step of converting the 5mC and 5hmC (or just the 5mC if the 5hmC is blocked) to 5 caC and/or 5fC. In some embodiments, this step comprises contacting the DNA or RNA sample with a ten eleven translocation (TET) enzyme. The TET enzymes are a family of enzymes that catalyze the transfer of an oxygen molecule to the C5 methyl group on 5mC resulting in the formation of 5-hydroxymethylcytosine (5hmC). TET further catalyzes the oxidation of 5hmC to 5fC and the oxidation of 5fC to form 5 caC. TET enzymes useful in the methods of the present disclosure include one or more of human TET1, TET2, and TET3; murine TET1, TET2, and TET3; Naegleria TET (NgTET); Coprinopsis cinerea (CcTET); the catalytic domain of mouse TET1 (mTET1CD); and derivatives or analogues thereof.

Methods of the present disclosure can also include the step of converting the 5 caC and/or 5fC in a nucleic acid sample to DHU. In some embodiments, this step comprises contacting the DNA or RNA sample with a reducing agent including, for example, a borane reducing agent such as pyridine borane, 2-picoline borane (pic-BH3), borane, sodium borohydride, sodium cyanoborohydride, sodium triacetoxyborohydride, triethylamine borane and tri(t-butyl)amine borane.

The present inventors have identified improved reaction conditions for conversion of 5fC and/or 5 caC to DHU. The improved reaction conditions unexpectedly increase conversion of 5fC and/or 5 caC to DHU while minimizing the false positive rate and bias while also providing for a shorter reaction time. In some preferred embodiments, the dimethylsulfoxide (DMSO) is included in the reaction mixture at a concentration of from 40.0% to 60.0% v/v and ranges and values therein (e.g., 41.0% to 59.0% v/v, 42.0% to 58.0% v/v, 43.0% to 57.0% v/v, 44.0% to 56.0% v/v, 45.0% to 55.0% v/v, 46.0% to 54.0% v/v, 47.0% to 53.0% v/v, 48.0% to 52.0% v/v, 49.0% to 59.0% v/v, 45.0% to 52.0% v/v, 46.0% to 52.0% v/v, or 47.0% to 52.0%) In some particularly preferred embodiments, the dimethylsulfoxide (DMSO) is included in the reaction mixture at a concentration of from 45.0% to 52.5% v/v. In some particularly preferred embodiments, the DMSO is included in the reaction mixture at a concentration of from 48.0% to 52.0% v/v. In some preferred embodiments, the reaction is conducted at a temperature of from 40.0 degrees Celsius to 60.0 degrees Celsius and ranges and values therein (e.g., (e.g., 41.0 to 59.0, 42.0 to 58.0, 43.0 to 57.0, 44.0 to 56.0, 45.0 to 55.0, 46.0 to 54.0%, 47.0 to 53.0, 48.0 to 52.0, 49.0 to 59.0, 45.0 to 52.0, 46.0 to 52.0, or 47.0 to 52.0 degrees Celsius). In some particularly preferred embodiments, the reaction is conducted at a temperature of from 45.0 degrees Celsius to 52.5 degrees Celsius. In some particularly preferred embodiments, the reaction is conducted at a temperature of from 48.0 degrees Celsius to 52.0 degrees Celsius. In some preferred embodiments, the reaction time for the borane reduction step is from 30 minutes to 90 minutes and ranges and values therein (e.g., 35 to 85, 40 to 80, 45 to 75, 50 to 70 or 50 to 60 minutes). In some particularly preferred embodiments, the reaction time for the borane reduction step is from 45 minutes to 75 minutes. In some particularly preferred embodiments, the reaction time for the borane reduction step is from 45 minutes to 60 minutes.

In some embodiments, the step of converting the 5hmC to 5fC comprises oxidizing the 5hmC to 5fC by contacting the DNA with, for example, manganese oxide (MnO₂), potassium ruthenate (K₂RuO₄) potassium perruthenate (KRuO₄) and/or Cu(II)/TEMPO (copper (II) perchlorate and 2,2,6,6-tetramethylpiperidine-1-oxyl (TEMPO)). The 5fC in the DNA sample is then converted to DHU by the methods disclosed herein (e.g., by the borane reaction).

Methods for Identifying 5mC. In some embodiments, the methods of the present disclosure include identifying 5mC in a DNA sample (targeted DNA or whole-genome), and providing a quantitative measure for the frequency of the 5mC modification at each location where the modification was identified in the DNA. In some embodiments, the percentages of the T at each transition location provide a quantitative level of 5mC at each location in the DNA. In accordance with these embodiments, methods for identifying 5mC can include the use of a blocking group. In other embodiments, methods for identifying 5mC do not require the use of a blocking group.

When a blocking group is used to identify 5mC in a DNA without including 5hmC, the 5hmC in the sample is blocked so that it is not subject to conversion to 5 caC and/or 5fC. In some embodiments, the 5hmC in the sample DNA are rendered non-reactive to the subsequent steps by adding a blocking group to the 5hmC. In one embodiment, the blocking group is a sugar, including a modified sugar, for example glucose or 6-azide-glucose (6-azido-6-deoxy-D-glucose). The sugar blocking group can be added to the hydroxymethyl group of 5hmC by contacting the DNA sample with uridine diphosphate (UDP)-sugar in the presence of one or more glucosyltransferase enzymes. In some embodiments, the glucosyltransferase is T4 bacteriophage β-glucosyltransferase (BGT), T4 bacteriophage α-glucosyltransferase (αGT), and derivatives and analogs thereof. BGT is an enzyme that catalyzes a chemical reaction in which a beta-D-glucosyl (glucose) residue is transferred from UDP-glucose to a 5-hydroxymethylcytosine residue in a nucleic acid.

Methods for Identifying 5hmC. In some embodiments, the methods of the present disclosure include identifying 5mC or 5hmC in a DNA sample (targeted DNA or whole-genome). In some embodiments, the method provides a quantitative measure for the frequency the of 5mC or 5hmC modifications at each location where the modifications were identified in the DNA. In some embodiments, the percentages of the T at each transition location provide a quantitative level of 5mC or 5hmC at each location in the DNA. In accordance with these embodiments, the method for identifying 5mC or 5hmC provides the location of 5mC and 5hmC, but does not distinguish between the two cytosine modifications. Rather, both 5mC and 5hmC are converted to DHU. The presence of DHU can be detected directly, or the modified DNA can be replicated, for instance by methods of the present disclosure, where the DHU is converted to T. In some embodiments, methods for identifying 5hmC include the use of a blocking group. In other embodiments, methods for identifying 5hmC do not require the use of a blocking group.

Methods for Identifying 5mC and/or Identifying 5hmC. The present disclosure provides a method for identifying 5mC and identifying 5hmC in a DNA by performing the method for identifying 5mC on a first DNA sample, and performing the method for identifying 5mC or 5hmC on a second DNA sample. In some embodiments, the first and second DNA samples are derived from the same DNA sample. For example, the first and second samples may be separate aliquots taken from a sample comprising DNA to be analyzed (e.g., cellular DNA or cfDNA).

Because the 5mC and 5hmC (that is not blocked) are converted to 5fC and 5 caC before conversion to DHU, any existing 5fC and 5 caC in the DNA sample will be detected as 5mC and/or 5hmC. However, given the extremely low levels of 5fC and 5 caC in genomic DNA under normal conditions, this will often be acceptable when analyzing methylation and hydroxymethylation in a DNA sample. The 5fC and 5 caC signals can be eliminated by protecting the 5fC and 5 caC from conversion to DHU by, for example, hydroxylamine conjugation and EDC coupling, respectively. In accordance with these embodiments, the method identifies the locations and percentages of 5hmC in the DNA through the comparison of 5mC locations and percentages with the locations and percentages of 5mC or 5hmC (together). Alternatively, the location and frequency of 5hmC modifications in a DNA can be measured directly.

In some embodiments, identifying 5fC and/or 5 caC provides the location of 5fC and/or 5 caC, but does not distinguish between these two cytosine modifications. Rather, both 5fC and 5 caC are converted to DHU, which is detected by the methods described herein.

Methods for Identifying 5 caC. In some embodiments, the method includes identifying 5 caC in a DNA sample (targeted DNA or whole-genome), and provides a quantitative measure for the frequency the of 5 caC modification at each location where the modification was identified in the DNA. In some embodiments, the percentages of the T at each transition location provide a quantitative level of 5 caC at each location in the DNA. In accordance with these embodiments, methods for identifying 5 caC can include the use of a blocking group. In other embodiments, methods for identifying 5 caC do not require the use of a blocking group.

In some embodiments, when the 5fC is blocked (and 5mC and 5hmC are not converted to DHU), the identification of 5 caC in the DNA can occur. In some embodiments, adding a blocking group to the 5fC in the DNA sample comprises contacting the DNA with an aldehyde reactive compound including, for example, hydroxylamine derivatives, hydrazine derivatives, and hydrazide derivatives. Hydroxylamine derivatives include ashydroxylamine; hydroxylamine hydrochloride; hydroxylammonium acid sulfate; hydroxylamine phosphate; O-methylhydroxylamine; O-hexylhydroxylamine; O-pentylhydroxylamine; O-benzylhydroxylamine; and particularly, O-ethylhydroxylamine (EtONH2), O-alkylated or O-arylated hydroxylamine, acid or salts thereof. Hydrazine derivatives include N-alkylhydrazine, N-arylhydrazine, N-benzylhydrazine, N,N-dialkylhydrazine, N,N-diarylhydrazine, N,N-dibenzylhydrazine, N,N-alkylbenzylhydrazine, N,N-arylbenzylhydrazine, and N,N-alkylarylhydrazine. Hydrazide e derivatives include-toluenesulfonylhydrazide, N-acylhydrazide, N,N-alkylacylhydrazide, N,N-benzylacylhydrazide, N,N-arylacylhydrazide, N-sulfonylhydrazide, N,N-alkylsulfonylhydrazide, N,N-benzylsulfonylhydrazide, and N,N-arylsulfonylhydrazide.

Methods for Identifying 5fC. In some embodiments, the method includes identifying 5fC in a DNA sample (targeted DNA or whole-genome), and provides a quantitative measure for the frequency the of 5fC modification at each location where the modification was identified in the DNA. In some embodiments, the percentages of the T at each transition location provide a quantitative level of 5fC at each location in the DNA. In accordance with these embodiments, methods for identifying 5fC can include the use of a blocking group. In other embodiments, methods for identifying 5fC do not require the use of a blocking group.

In some embodiments, adding a blocking group to the 5 caC in the DNA sample can be accomplished by (i) contacting the DNA sample with a coupling agent, for example a carboxylic acid derivatization reagent like carbodiimide derivatives such as 1-ethyl-3-(3-dimethylaminopropyl) carbodiimide (EDC) or N,N′-dicyclohexylcarbodiimide (DCC), and (ii) contacting the DNA sample with an amine, hydrazine or hydroxylamine compound. Thus, for example, 5 caC can be blocked by treating the DNA sample with EDC and then benzylamine, ethylamine, or another amine to form an amide that blocks 5 caC from conversion to DHU (e.g., by borane reduction).

### 3. SEQUENCING LIBRARIES

The present disclosure provides a method of obtaining a methylation signature. In some embodiments, the method includes isolating DNA (e.g., cellular or cfDNA) from a sample; preparing a sequencing library comprising the DNA; and performing TET-assisted Pyridine Borane Sequencing (TAPS) on the sequencing library to obtain a methylation signature of the DNA. In some embodiments, the methylation signature is a whole-genome methylation signature.

In some embodiments, preparing the sequencing library comprises ligating sequencing adapters to the isolated DNA to facilitate performing a sequencing reaction. Suitable sequencing adapters for massively parallel sequencing technologies may be utilized. The present invention is not limited to any particular sequencing technology. In some preferred embodiments, sequencing technologies such as those provided by Illumina or Nanopore may be utilized. For example, suitable sequencing technologies for use in the present invention include, but are not limited, to those described in US Pat. Publ. 20100120098, US Pat. Publ. 20120208705, US Pat. Publ. 20120208724, WO2012/061832, and US Pat. Publ. 2015/0368638, each of which is incorporated herein by reference in its entirety.

In some embodiments, the adapter comprises one or more sites that can hybridize to a primer. In some embodiments, an adapter comprises at least a first primer site. In some embodiments, an adapter comprises at least a first primer site and a second primer site. The orientation of the primer sites in such embodiments can be such that a primer hybridizing to the first primer site and a primer hybridizing to the second primer site are in the same orientation, or in different orientations. In one embodiment, the primer sequence in the linker can be complementary to a primer used for amplification. In another embodiment, the primer sequence is complementary to a primer used for sequencing.

In some embodiments, a linker can include a first primer site, a second primer site having a non-amplifiable site disposed therebetween. The non-amplifiable site is useful to block extension of a polynucleotide strand between the first and second primer sites, wherein the polynucleotide strand hybridizes to one of the primer sites. The non-amplifiable site can also be useful to prevent concatamers. Examples of non-amplifiable sites include a nucleotide analogue, non-nucleotide chemical moiety, amino-acid, peptide, and polypeptide. In some embodiments, a non-amplifiable site comprises a nucleotide analogue that does not significantly basepair with A, C, G or T.

Some embodiments include a linker comprising a first primer site, a second primer site having a fragmentation site disposed therebetween. Other embodiments can use a forked or Y-shaped adapter design useful for directional sequencing, as described in U.S. Pat. No. 7,741,463, which is incorporated herein by reference.

In some embodiments, the adapter may comprise an index or barcode sequence. In further preferred embodiments, the adapter may comprise a Unique Molecular Identifier (UMI).

In some embodiments, carrier nucleic acids or a mix of carrier nucleic acids (e.g., DNA) are added to the sequencing library prior to performing TAPS. Carrier nucleic acids can be any specific or non-specific DNA molecules (or nucleic acid derivatives thereof) that enhance one or more aspects of DNA recovery from a sample.

As indicated above, in some preferred embodiments, nucleic acids containing DHU residues are subjected to a complementary strand synthesis step with a first polymerase or polymerase mixture that is tolerant of DHU residues and/or products resulting from the introduction of the DHU residues and/or the TAPS process and an exponential amplification step with a second polymerase or polymerase mixture. In some embodiments, the second polymerase or polymerase mixture may be the same as the first polymerase or polymerase mixture or may be a different polymerase or polymerase mixture than the first. In certain embodiments where the second polymerase mixture is different than the first, the second polymerase or polymerase mixture may also be tolerant of DHU residues and/or products resulting from the introduction of the DHU residues into the target nucleic acid molecule and/or the TAPS process. In embodiments where the same DHU and/or TAPS tolerant polymerase is used for both complementary strand synthesis and exponential amplification, it will be understood that the initial complementary strand synthesis step may be part of the exponential amplification process. The complementary strand synthesis step and amplification steps may be performed before or after incorporation of the sequencing adapter sequences onto the target nucleic acids. In some preferred embodiments, the first polymerase or polymerase mixture is characterized in having an error rate of greater than 5.0×10⁻⁵. In some preferred embodiments, the complementary strand synthesis step with the DHU and/or TAPS tolerant polymerase or polymerase mixture results in improved coverage of highly methylated (and thus DHU-rich) regions as compared to the same protocol without the complementary strand synthesis step with the DHU and/or TAPS tolerant polymerase or polymerase mixture.

Suitable DHU and/or TAPS first polymerase and polymerase mixtures for use in the complementary strand synthesis step include, but are not limited to Bst 3.0 polymerase (New England Biolabs, Beverly, MA), Sulfolobus DNA Polymerase IV (New England Biolabs, Beverly, MA), a combination of Bst3.0 polymerase and Sulfolobus DNA Polymerase IV, Klenow polymerase (New England Biolabs, Beverly, MA), Klenow exo-polymerase (ThermoFisher Scientific, Grand Island, NY), Polk polymerase, Mu-mLV reverse transcriptase, SD polymerase (Bioron), Tth polymerase (SigmaAldrich, St. Louis, MO), OneTaq™ (New England Biolabs, Beverly, MA), a combination of OneTaq™ (New England Biolabs, Beverly, MA) and Tth polymerase, 5D4 polymerase and a 5D4 polymerase blend with Taq polymerase. In some preferred embodiments, the first polymerase or polymerase mixture is thermolabile. In other preferred embodiments, the first polymerase or polymerase mixture is thermostable.

The polymerase utilized for the exponential amplification step may be any polymerase suited for use in amplification and/or sequencing and may be the same or different as the first polymerase. In some preferred embodiments, the polymerase used in the exponential amplification is different from the first polymerase and denoted as a second polymerase or polymerase mixture. In some particularly preferred embodiments, the polymerase used for the exponential amplification step has an error rate that is less than the first polymerase or polymerase mixture. In some preferred embodiments, the second polymerase or polymerase mixture is characterized as a high-fidelity polymerase. In some preferred embodiments, the second polymerase or polymerase mixture is characterized in having an error rate of less than 5.0×10⁻⁵. In some preferred embodiments, the second polymerase or polymerase mixture is selected from Taq polymerases such as GoTaq™ polymerase (Promega, Fitchburg, WI) and engineered B family polymerases such as KAPA HiFi Uracil+ polymerase (Roche, Indianapolis, IN). In some preferred embodiments, the first polymerase or polymerase mixture is thermostable.

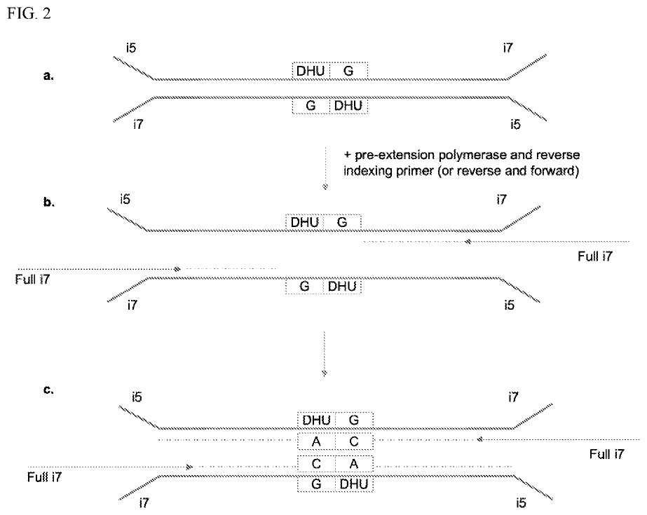

In some preferred embodiments, the complementary strand synthesis step utilizes forward and/or reverse primers that anneal to the sequencing adapter. In some preferred embodiments, the exponential amplification step utilizes sequencing primers that anneal to a region of the sequencing adapter. See FIG. 2.

It is contemplated that DNA methylation signatures are useful for understanding basic biological processes and disease pathology as well as for disease detection. For example, methylation signatures/frequencies/markers etc. can be useful in understanding and studying gene regulation, genomic imprinting, differentiation, development, gene-environment interaction (e.g., smoking, nutrition), aging, numerous diseases and conditions (e.g., auto-immune diseases, cancer, cardiovascular diseases, CNS diseases, congenital diseases, infectious diseases, metabolic diseases and status, NIPT-related testing, etc.), for detecting and diagnosing cancer and other diseases and for monitoring transplants. In some embodiments, and as described herein, the method further comprises identifying at least one methylation biomarker from the DNA methylation signature (such as a whole-genome DNA methylation signature) and determining if the methylation biomarker differs from the methylation biomarker in a reference or control sequence. In some embodiments, the methylation biomarker comprises a differentially methylated region (DMR). In some embodiments, the method further comprises classifying the sample based on the DMR as compared to a reference DMR. In some embodiments, the reference DMR corresponds to a non-disease control, or a disease control.

In some embodiments, and as described herein, the method further comprises identifying at least one methylation biomarker from the DNA methylation signature, and determining a tissue-of-origin corresponding to the methylation biomarker. In some embodiments, the method further comprises classifying the sample based on the tissue-of-origin biomarker.

In some embodiments, and as described herein, the method further comprises identifying a DNA fragmentation profile, and determining whether the fragmentation profile is indicative of cancer. In accordance with these embodiments, DNA fragmentation profile can be determined from TAPS sequencing data (e.g., read pair alignment positions).

In some embodiments, the method further comprises identifying at least one sequence variant in the DNA sample, and determining whether the sequence variant is indicative of cancer. For example, in some embodiments, TAPS can also differentiate methylation from C-to-T genetic variants or single nucleotide polymorphisms (SNPs), and therefore, can be used to detect genetic variants. In some embodiments, methylations and C-to-T SNPs can result in different patterns in TAPS. For example, methylations can result in T/G reads in an original top strand/original bottom strand, and A/C reads in strands complementary to these. In some embodiments, C-to-T SNPs can result in T/A reads in an original top strand/original bottom strand and strands complementary to these. This further increases the utility of TAPS in providing both methylation information and genetic variants, and therefore mutations, in one experiment and sequencing run. This ability of the TAPS methods disclosed herein provides integration of genomic analysis with epigenetic analysis, and a substantial reduction of sequencing cost by eliminating the need to perform, for example, standard whole genome sequencing (WGS).

In accordance with the above embodiments, methods of the present disclosure include the use of TAPS to generate information pertaining to methylation signatures, methylation biomarkers, DNA fragment profiles, DNA sequence information (e.g., variants), and tissue-of-origin information in a single experiment to diagnose/detect a disease or other condition (e.g., those provided as examples above) in a subject. As would be recognized by one of ordinary skill in the art based on the present disclosure, TAPS as disclosed herein can be used to generate any combination of methylation signatures, methylation biomarkers, DNA fragment profiles, DNA sequence information (e.g., variants), and tissue-of-origin information to diagnose/detect a disease or other condition (e.g., those provided as examples above) in a subject. In some embodiments, a methylation signature can be obtained, and one or more of a methylation biomarker, a DNA fragment profile, DNA sequence information (e.g., variants), and tissue-of-origin information can also be obtained and used to diagnose/detect a disease or other condition (e.g., those provided as examples above) in a subject. In some embodiments, the methylation status of a biomarker can be obtained, and one or more of a methylation signature, a DNA fragment profile, DNA sequence information (e.g., variants), and tissue-of-origin information can also be obtained and used to diagnose/detect a disease or other condition (e.g., those provided as examples above) in a subject. In some embodiments, a DNA fragmentation profile can be obtained, and one or more of a methylation signature, a methylation biomarker, DNA sequence information (e.g., variants), and tissue-of-origin information can also be obtained and used to diagnose/detect a disease or other condition (e.g., those provided as examples above) in a subject. In some embodiments, a DNA sequence variant can be identified, and one or more of a methylation signature, a methylation biomarker, a DNA fragment profile, and tissue-of-origin information can also be obtained and used to diagnose/detect a disease or other condition (e.g., those provided as examples above) in a subject. In some embodiments, tissue-of-origin information can be obtained (e.g., from a whole genome DNA methylation signature), and one or more of the methylation signature, a methylation biomarker, a DNA fragment profile, and DNA sequence information (e.g., variants), can also be obtained and used to diagnose/detect a disease or other condition (e.g., those provided as examples above) in a subject.

In some embodiments, performing TAPS on the sequencing library to obtain the whole-genome methylation signature comprises identifying 5mC modifications in the DNA and providing a quantitative measure for frequency of the 5mC modifications. In some embodiments, performing TAPS on the sequencing library to obtain the whole-genome methylation signature comprises identifying 5hmC modifications in the DNA and providing a quantitative measure for frequency of the 5hmC modifications. In some embodiments, performing TAPS on the sequencing library to obtain the whole-genome methylation signature comprises identifying 5 caC modifications in the DNA and providing a quantitative measure for frequency of the 5 caC modifications. In some embodiments, performing TAPS on the sequencing library to obtain the whole-genome methylation signature comprises identifying 5fC modifications in the DNA and providing a quantitative measure for frequency of the 5fC modifications.

As would be recognized by one of ordinary skill in the art based on the present disclosure, the methods described herein (e.g., TAPS) can be used to diagnose/detect any type of cancer. Types of cancers that can be detected/diagnosed using the methods of the present disclosure include, but are not limited to, lung cancer, melanoma, colon cancer, colorectal cancer, neuroblastoma, breast cancer, prostate cancer, renal cell cancer, transitional cell carcinoma, cholangiocarcinoma, brain cancer, non-small cell lung cancer, pancreatic cancer, liver cancer, gastric carcinoma, bladder cancer, esophageal cancer, mesothelioma, thyroid cancer, head and neck cancer, osteosarcoma, hepatocellular carcinoma, carcinoma of unknown primary, ovarian carcinoma, endometrial carcinoma, glioblastoma, Hodgkin lymphoma and non-Hodgkin lymphomas. In some embodiments, types of cancers or metastasizing forms of cancers that can be detected/diagnosed by the methods of the present disclosure include, but are not limited to, carcinoma, sarcoma, lymphoma, germ cell tumor and blastoma. In some embodiments, the cancer is invasive and/or metastatic cancer (e.g., stage II cancer, stage III cancer or stage IV cancer). In some embodiments, the cancer is an early-stage cancer (e.g., stage 0 cancer, stage I cancer), and/or is not invasive and/or metastatic cancer.

In accordance with these embodiments, the present disclosure provides methods for identifying the location of one or more of 5mC, 5hmC, 5 caC and/or 5fC in a nucleic acid quantitatively with base-resolution without affecting the unmodified cytosine. In some embodiments, the nucleic acid is DNA. In some embodiments, the DNA is cfDNA (e.g., circulating cfDNA). In some embodiments, the nucleic acid is RNA. In some embodiments, a nucleic acid sample comprises a target nucleic acid that is DNA or a target nucleic acid that is RNA. In some embodiments, the methods are applied to a whole genome, and not limited to a specific target nucleic acid.

The nucleic acid may be any nucleic acid having cytosine modifications (i.e., 5mC, 5hmC, 5fC, and/or 5 caC) but not limited to, DNA fragments and/or genomic DNA. The nucleic acid can be a single nucleic acid molecule in the sample, or may be the entire population of nucleic acid molecules in a sample, or any portion thereof (whole genome or a subset thereof). The nucleic acid can be the native nucleic acid from the source (e.g., cells, tissue samples, etc.) or can pre-converted into a high-throughput sequencing-ready form, for example by fragmentation, repair and ligation with adapters for sequencing. Thus, nucleic acids can comprise a plurality of nucleic acid sequences such that the methods described herein may be used to generate a library of target nucleic acid sequences that can be analyzed individually (e.g., by determining the sequence of individual targets) or in a group (e.g., by high-throughput or next generation sequencing methods).

Because the methods of the present disclosure utilize mild enzymatic and chemical reactions that avoid the substantial degradation of nucleic acids associated with methods like bisulfite sequencing, the methods of the present disclosure are useful in analysis of low-input samples, such as circulating cell-free DNA and in single-cell analysis.

In some embodiments, the DNA sample comprises picogram quantities of DNA. In some embodiments, the DNA sample comprises from about 1 pg to about 900 pg DNA, from about 1 pg to about 500 pg DNA, from about 1 pg to about 100 pg DNA, from about 1 pg to about 50 pg DNA, or from about 1 to about 10 pg DNA. In some embodiments, the DNA sample comprises less than about 200 pg, less than about 100 pg DNA, less than about 50 pg DNA, less than about 20 pg DNA, less than about 15 pg DNA, less than about 10 pg DNA, or less than about 5 pg DNA.

In some embodiments, the DNA sample comprises nanogram quantities of DNA. The sample DNA for use in the methods of the present disclosure can be any quantity including, but not limited to, DNA from a single cell or bulk DNA samples. In some embodiments, the methods can be performed on a DNA sample comprising from about 1 to about 500 ng of DNA, from about 1 to about 200 ng of DNA, from about 1 to about 100 ng of DNA, from about 1 to about 50 ng of DNA, from about 1 to about 10 ng of DNA, from about 2 to about 5 ng of DNA. In some embodiments, the DNA sample comprises less than about 100 ng of DNA, less than about 50 ng of DNA, less than 40 ng of DNA, less than 30 ng of DNA, less than 20 ng of DNA, less than 15 ng of DNA, less than 5 ng of DNA, and less than 2 ng of DNA. In some embodiments, the DNA sample comprises microgram quantities of DNA.

The methods of the present disclosure can also include the step of amplifying the copy number of a modified nucleic acid by methods known in the art. When the modified nucleic acid is DNA, the copy number can be increased by, for example, PCR, cloning, and primer extension. The copy number of individual target DNAs can be amplified by PCR using primers specific for a particular target DNA sequence. Alternatively, a plurality of different modified target DNA sequences can be amplified by cloning into a DNA vector by standard techniques. In some embodiments, the copy number of a plurality of different modified target DNA sequences is increased by PCR to generate a library for next generation sequencing where, e.g., double-stranded adapter DNA has been previously ligated to the sample DNA (or to the modified sample DNA) and PCR is performed using primers complimentary to the adapter DNA.

In some embodiments, the method comprises the step of detecting the sequence of the modified nucleic acid. The modified target DNA or RNA contains DHU at positions where one or more of 5mC, 5hmC, 5fC, and 5 caC were present in the unmodified target DNA or RNA. DHU acts as a T in DNA replication and sequencing methods. Thus, the cytosine modifications can be detected by any direct or indirect method that identifies a C to T transition known in the art. Such methods include sequencing methods such as Sanger sequencing, microarray, and next generation sequencing methods. The C to T transition can also be detected by restriction enzyme analysis where the C to T transition abolishes or introduces a restriction endonuclease recognition sequence.

Embodiments of the present disclosure also provide kits for identification of 5mC and 5hmC in a DNA. Such kits comprise reagents for identification of 5mC and 5hmC by the methods described herein. The kits may also contain the reagents for identification of 5 caC and for the identification of 5fC by the methods described herein. In some embodiments, the kit comprises a TET enzyme, a borane reducing agent and instructions for performing the method. In some embodiments, the borane reducing agent is selected from one or more of the group consisting of pyridine borane, 2-picoline borane (pic-BH3), borane, sodium borohydride, sodium cyanoborohydride, and sodium triacetoxyborohydride. In further preferred embodiments, the kits comprise first and second polymerases or polymerase mixtures as described in detail above.

In some embodiments, the kit further comprises a 5hmC blocking group and a glucosyltransferase enzyme. In some embodiments, the blocking group added to 5hmC is a sugar. In some embodiments, the sugar is a naturally-occurring sugar or a modified sugar, for example glucose or a modified glucose. In some embodiments, the blocking group is added to 5hmC by contacting a nucleic acid sample with UDP linked to a sugar, for example UDP-glucose or UDP linked to a modified glucose in the presence of a glucosyltransferase enzyme, for example, T4 bacteriophage β-glucosyltransferase (BGT) and T4 bacteriophage α-glucosyltransferase (αGT) and derivatives and analogs thereof.

In some embodiments, the kit further comprises an oxidizing agent selected from manganese oxide (MnO₂), potassium ruthenate (K₂RuO₄), potassium perruthenate (KRuO4) and/or Cu(II)/TEMPO (copper (II) perchlorate and 2,2,6,6-tetramethylpiperidine-1-oxyl (TEMPO)). In some embodiments, the kit comprises reagents for blocking 5fC in the nucleic acid sample. In some embodiments, the kit comprises an aldehyde reactive compound including, for example, hydroxylamine derivatives, hydrazine derivatives, and hydrazide derivatives as described herein. In some embodiments, the kit comprises reagents for blocking 5 caC as described herein. In some embodiments, the kit comprises reagents for isolating DNA or RNA. In some embodiments the kit comprises reagents for isolating low-input DNA from a sample, for example cfDNA from blood, plasma, or serum.

In some embodiments, the methods of the present disclosure include treating a patient (e.g., a patient with cancer, with early-stage cancer, or who is suspected of having cancer). In some embodiments, the methods include determining a methylation signature as provided herein and administering a treatment to a patient based on the results of determining the methylation signature. The treatment can include administration of a pharmaceutical compound, a vaccine, performing a surgery, imaging the patient, and/or performing another test. In some embodiments, the methods of the present disclosure can be used as part of clinical screening, a method of prognosis assessment, a method of monitoring the results of therapy, a method to identify patients most likely to respond to a particular therapeutic treatment, a method of imaging a patient or subject, and a method for drug screening and development.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

NGS libraries generated from TET-assisted pyridine borane sequencing (TAPS) show reduced coverage in regions of DNA with a high density of methylated cytosines relative to average coverage (FIG. 1). In FIG. 1, the normalized GC bias metric for a reference fully methylated lambda DNA sequence serves as a surrogate for highly methylated DNA and the difference in the curves for the TAPS-treated and non-TAPS-treated fully methylated lambda DNA is representative of differences in coverage of methylated regions that would be seen in in a biological sample. Underrepresentation in clinically relevant regions of a methylated biological sample results in reduced sensitivity and necessitates increased sequencing to obtain sufficient coverage. In order to improve the sequencing efficiency of TAPS libraries, we have discovered that an initial complementary strand synthesis step with polymerases that are tolerant of DHU or other TAPS products improves the coverage in highly methylated regions of DNA, as measured by normalized GC bias, when combined with typical library amplification techniques.

Partial NGS sequencing library adapters (partial Y-shaped i5 and i7 adapters shown in green in FIG. 2) are ligated onto fragmented DNA either before or after TAPS treatment. During TAPS treatment, TET oxidation and borane reduction converts methylated cytosines (meC) to dihydrouracil (DHU) bases (shown in purple boxes in FIG. 2).

During amplification of these libraries with indexing primers, polymerases insert an adenine opposite a DHU base resulting in subsequent conversion to thymidine in the following amplification step. However, many polymerases disfavor replication opposite DHU bases or products resulting from the introduction of the DHU residues and/or the TAPS process.

In the complementary strand synthesis step this initial replication past DHU bases is undertaken with alternate polymerases. For partial adapters as shown in FIG. 2, the complementary strand synthesis step utilizes one of a number of polymerases found to have less bias against DHU e.g., Bst 3.0 as well as the reverse primer (full i7 as shown in FIG. 2b), allowing copying of the library molecule with an incubation at a constant temperature (with or without an initial denaturation step). This results in adapter-ligated DNA with the DHU bases now converted to adenine (FIG. 2c). This DNA then undergoes typical PCR amplification with a very high-fidelity polymerase with excellent GC bias coverage such as Kapa Hifi Uracil+, with the addition of forward indexing primer and library amplification primers to generate enough DNA for sequencing. We have also found that a synthesis step utilizing both indexing primers (full length i5 and i7) achieves the same outcome. It is expected that similar conditions would be beneficial for amplifying libraries generated with full length adapter ligated DNA.

Exemplary reagents utilized in the sequencing methods described herein are provided in Tables 1 to 3:

TABLE 1
Complementary strand synthesis reaction reagents

| | | |
|---|---|---|
| | Separate buffer | Spike in |
| Reagent | option | option |

| DNA | x | μl | x | μl |
|---|---|---|---|---|
| Buffer specific for | 5 | μl | — | |
| polymerase | | | | |

| | | |
|---|---|---|
| dNTPs | If required | — |

| | | | | |
|---|---|---|---|---|
| Primers* | 1-10 | μl | 1-10 | μl |

| | | | |
|---|---|---|---|
| Kapa Hifi U+ | — | 25 | μl |

| | | | | |
|---|---|---|---|---|
| mastermix | | | | |
| Polymerase⁺ | 1-5 | μl | 1-5 | μl |

| | | |
|---|---|---|
| Water | To 50 μl | To 50 μl |

*primers = full reverse i7, or full reverse i7 and forward i5
⁺thermolabile polymerases must be added after denaturing step if denaturing step is included

TABLE 2
Complementary strand synthesis incubation steps

| Steps | Temperature | Time | Cycles⁺ |
|---|---|---|---|
| Denaturing | 70-98° C. | 1-5 mins | 1-4 cycles |
| (optional) | | | tested |
| Anneal/extension | 37-65° C.* | 15-240 mins | |

*temperature depending on polymerase
⁺typically 1 cycle but 2-4 cycles also tested

TABLE 3
Subsequent processing

| | | Further | Other | |
|---|---|---|---|---|
| | AMPure bead | primer | reagent | PCR |
| Steps | clean up | addition* | addition | amplification |
| Separate | optional | i5 and library | Kapa Hifi | Yes |
| polymerase | | amplification | Uracil+ | |
| buffer option | | | | |
| Spike in | Not required | i5 and library | — | Yes |
| option | | amplification | | |

*if full reverse i7 and forward i5 used in complementary strand synthesis step, no additional primers are required

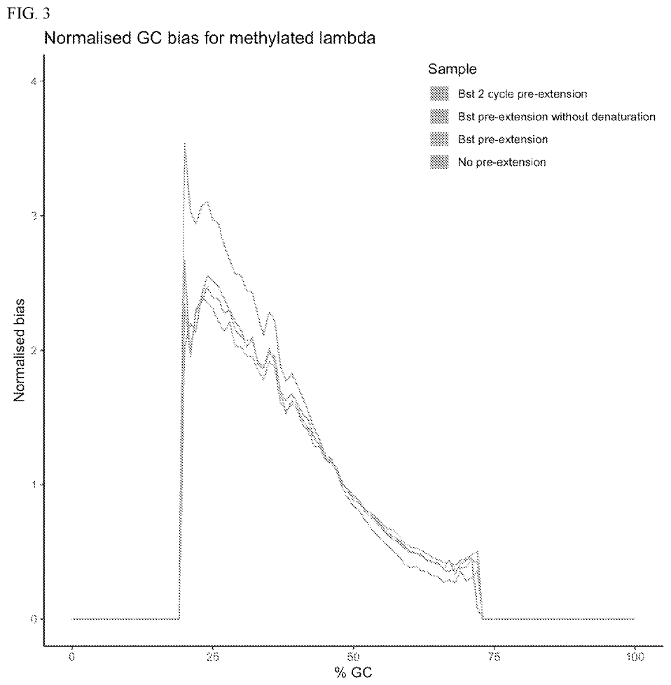

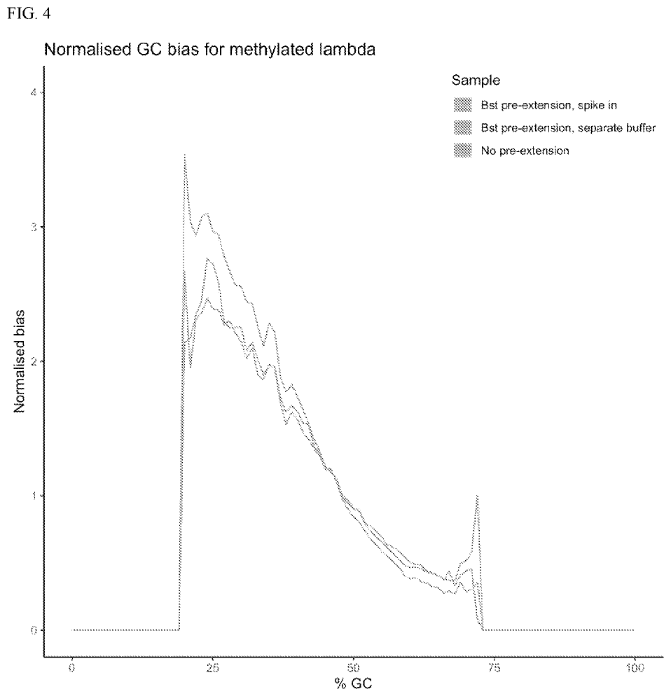

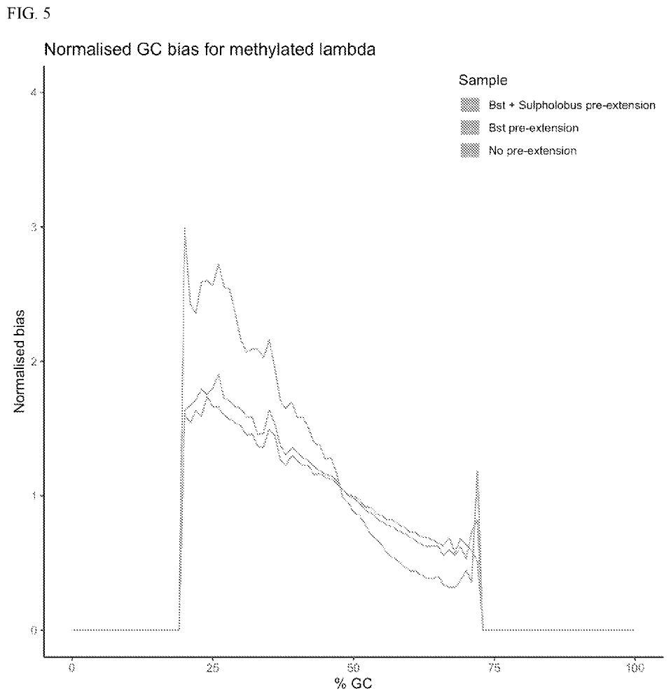

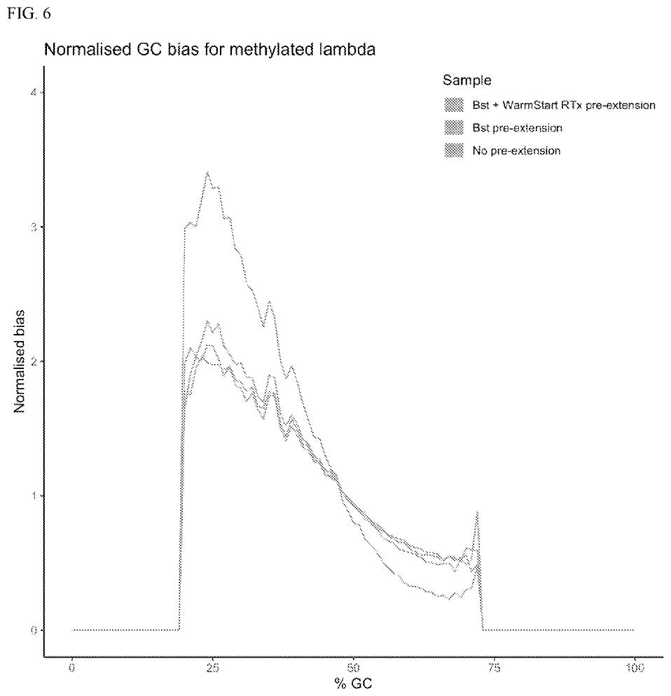

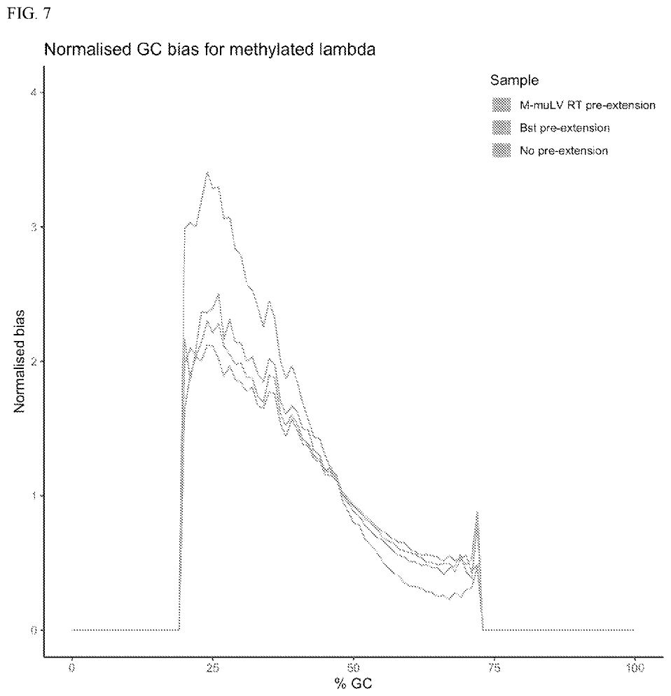

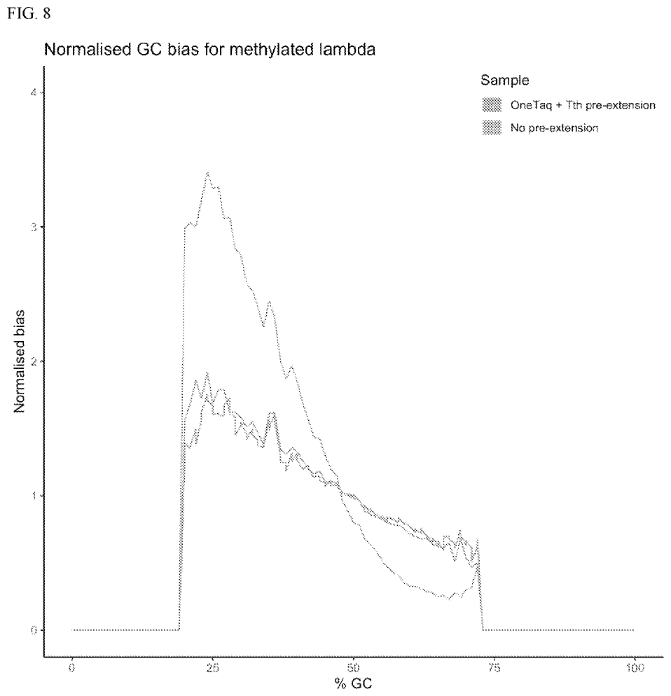

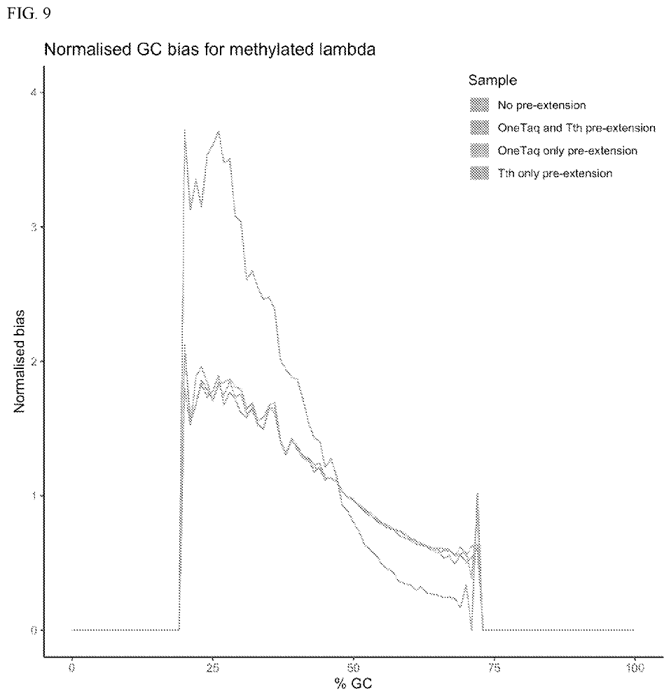

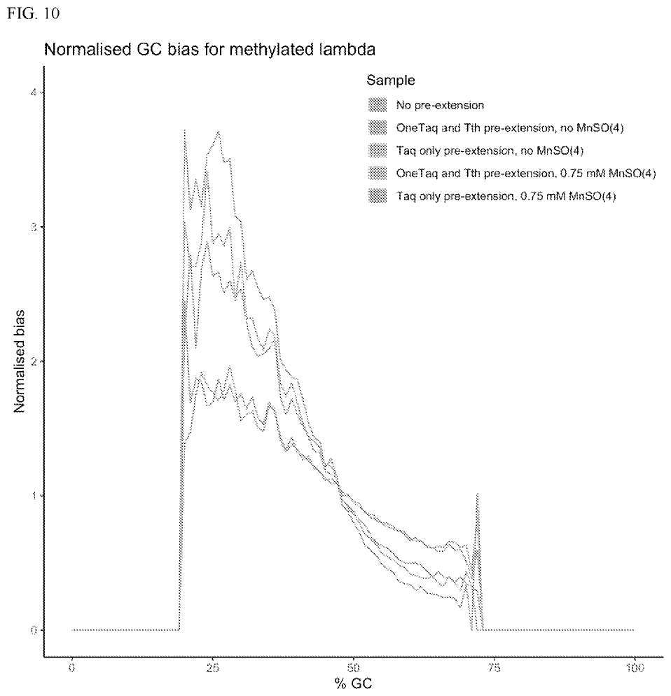

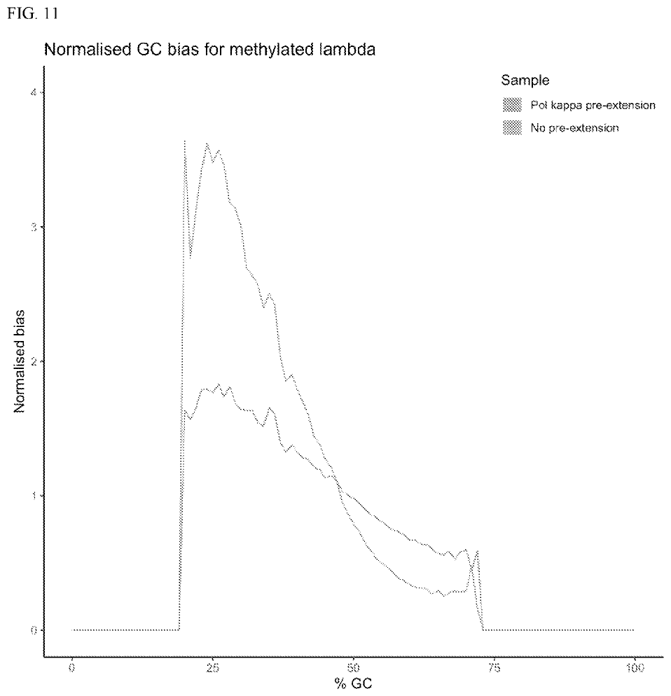

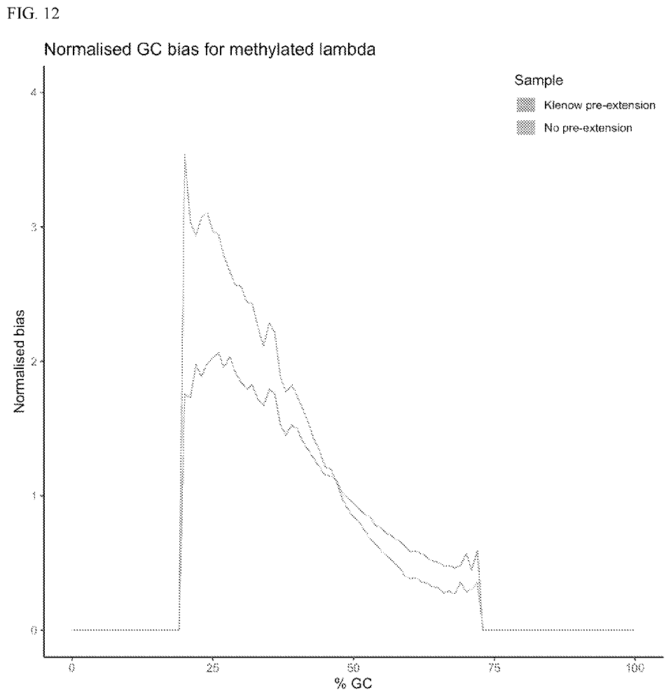

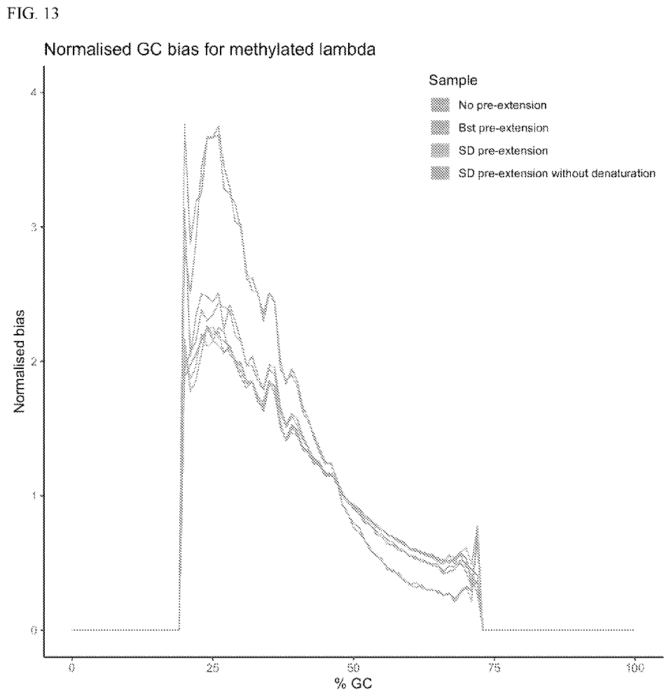

FIGS. 3 to 14 provide results of sequencing a fully methylated lambda spike in prepared with Kapa Hyperprep™ and a variety of polymerases in the complementary strand synthesis and amplification steps. Bst 3.0 polymerase shows improved coverage uniformity with various workflow options and in combination with several other polymerases and reverse transcriptases. FIG. 8 shows improvement with OneTaq™ and Tth in the presence of OneTaq buffer and MnSO₄. We have further optimized this method and shown the same improvement with only OneTaq, only Tth or only Taq polymerase, and MnSO₄ of which 0.5-0.75 mM was found to be optimal (FIGS. 9 and 10). Unless otherwise stated, OneTaq and Tth conditions have 0.75 mM MnSO₄. FIGS. 11, 12, and 13 show improvement using polymerase κ, Klenow exo-, and SD polymerase, respectively.

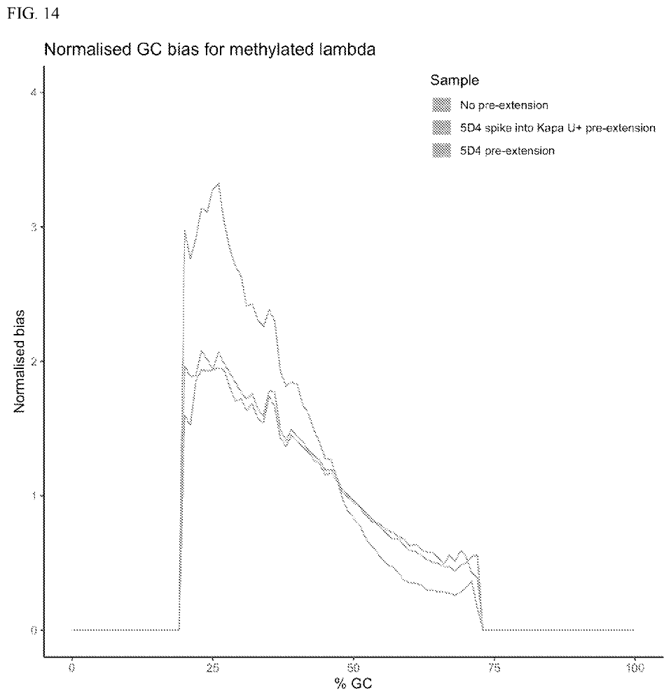

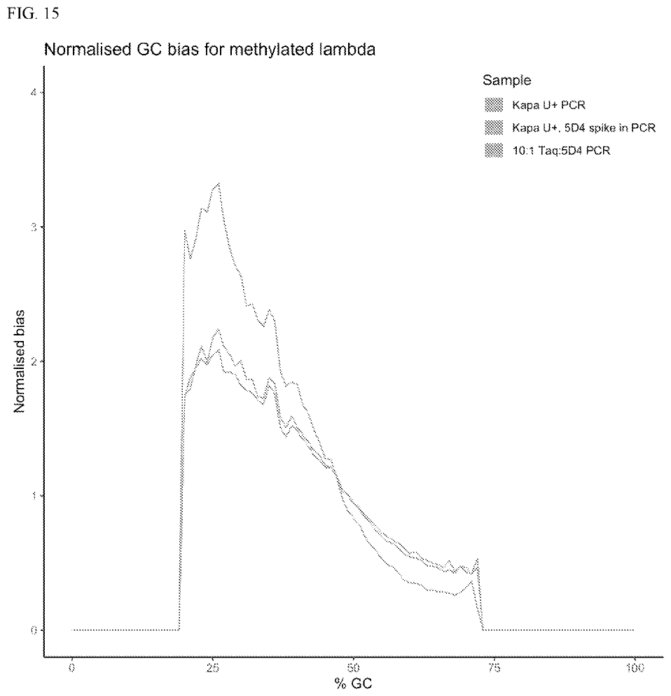

We have also found that an engineered polymerase, 5D4, performs well in complementary strand synthesis step conditions either alone, or as a spike into Kapa Hifi Uracil+ (FIG. 14). We note that 5D4 also improves coverage of high DHU region when used in library amplification (without an initial complementary strand synthesis step) in combination with Taq polymerase or as a spike into Kapa Hifi Uracil+ (FIG. 15).

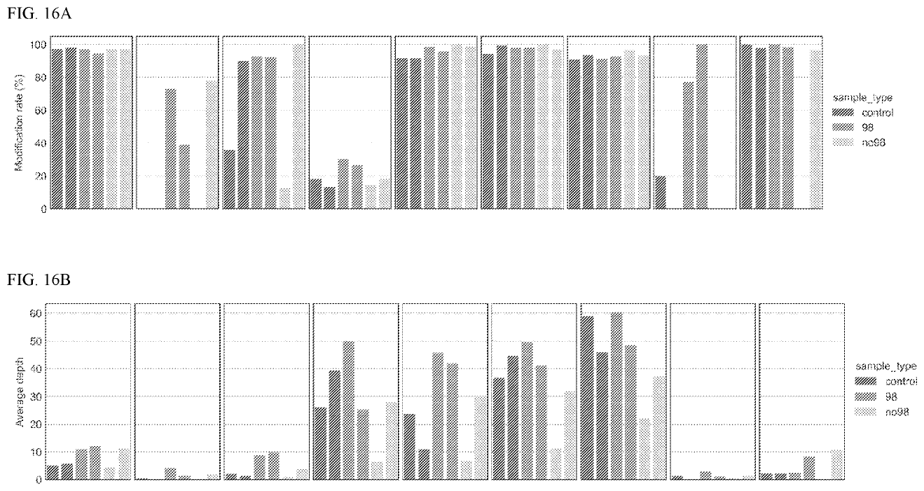

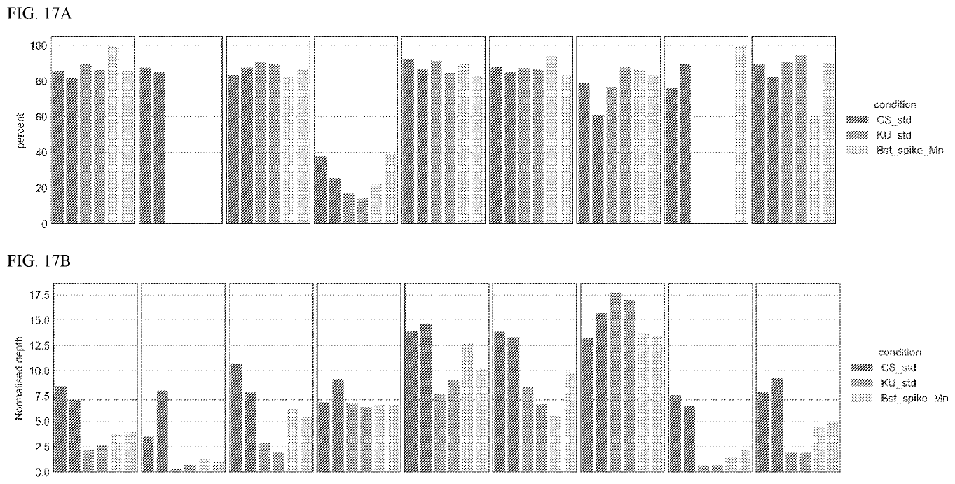

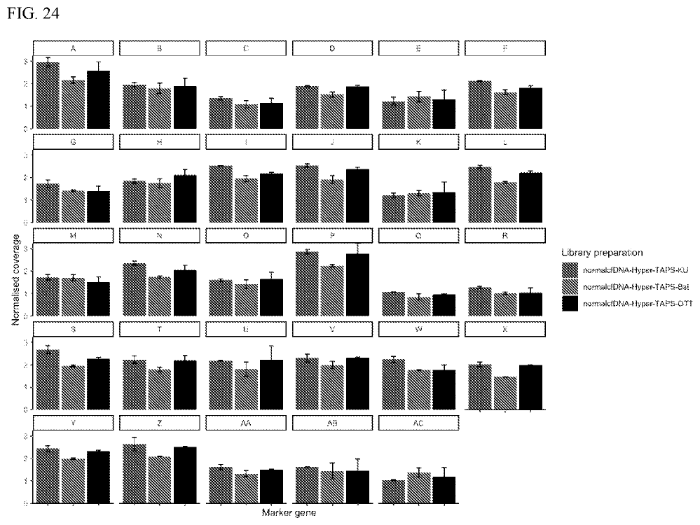

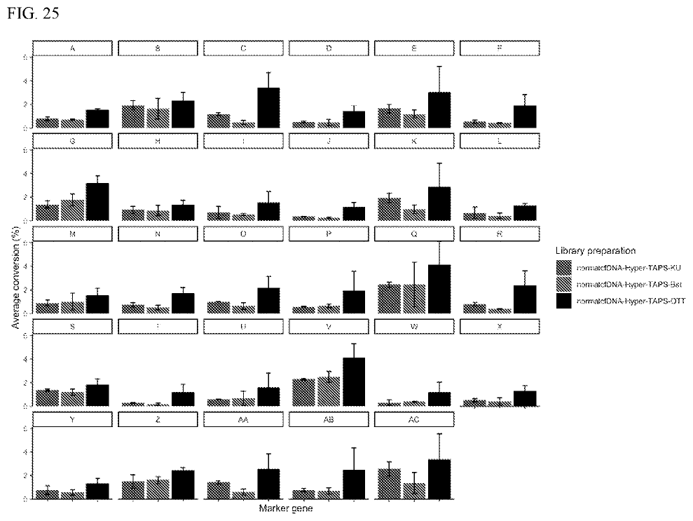

We have further found that selected beneficial complementary strand synthesis conditions lead to improved coverage of marker regions, in high coverage whole genome sequencing, that are typically highly methylated and/or have a high density of CpG sites leading to lower than average coverage when sequencing under standard conditions. Shown here are Bst3.0 complementary strand synthesis step conditions (FIG. 16A-B) and OneTaq™ and Tth complementary strand synthesis step conditions (FIG. 17A-B). We also see benefit where there is competition between methylated and non-methylated versions of a target sequence. This manifests as an improved methylation signal in markers that are methylated to a lesser extent. See FIGS. 24 and 25. FIG. 24 shows normalized coverage in selected marker regions with low levels of methylation following TAPS and amplification with different polymerases (Kapa Hifi Uracil+, Bst and OTT). FIG. 25 shows conversion rates in selected marker regions following TAPS and amplification with different polymerases (Kapa Hifi Uracil+, Bst and OTT).

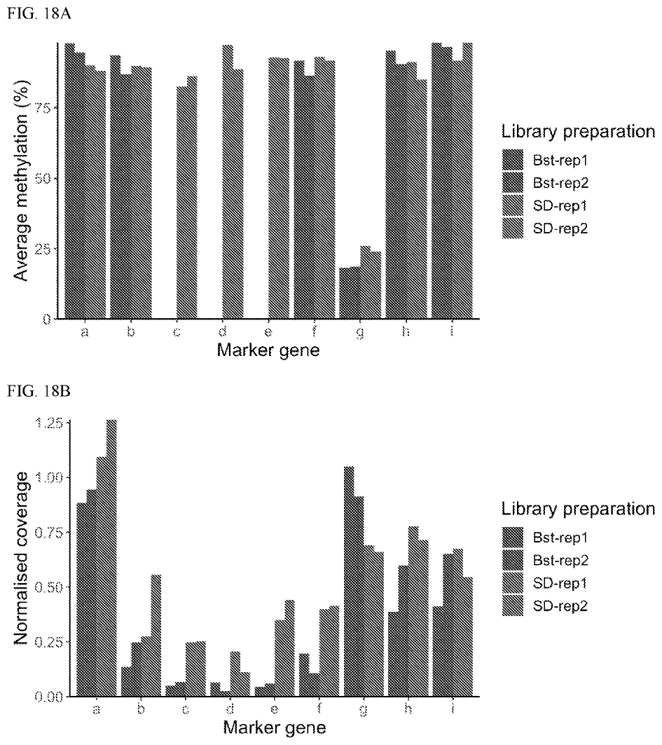

Initial complementary strand synthesis with SD polymerase also shows similar or greater normalized coverage compared to a Bst complementary strand synthesis step on highly methylated marker regions in Whole Genome Sequencing of NA12878 (FIG. 18A-B). The missing datapoints for the methylation of some regions highlights the low coverage of these highly methylated regions and in this instance although improved coverage relative to no initial complementary strand synthesis step, there were not enough reads to confidently determine average methylation.

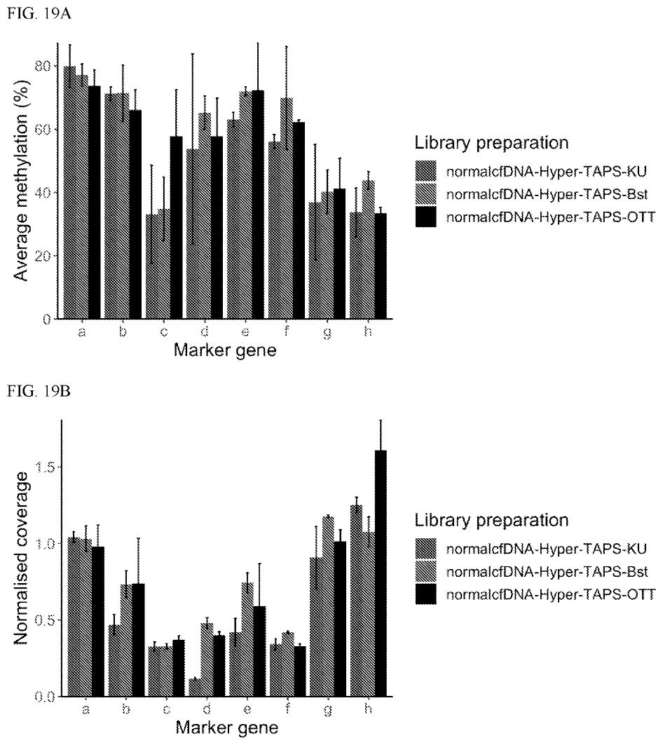

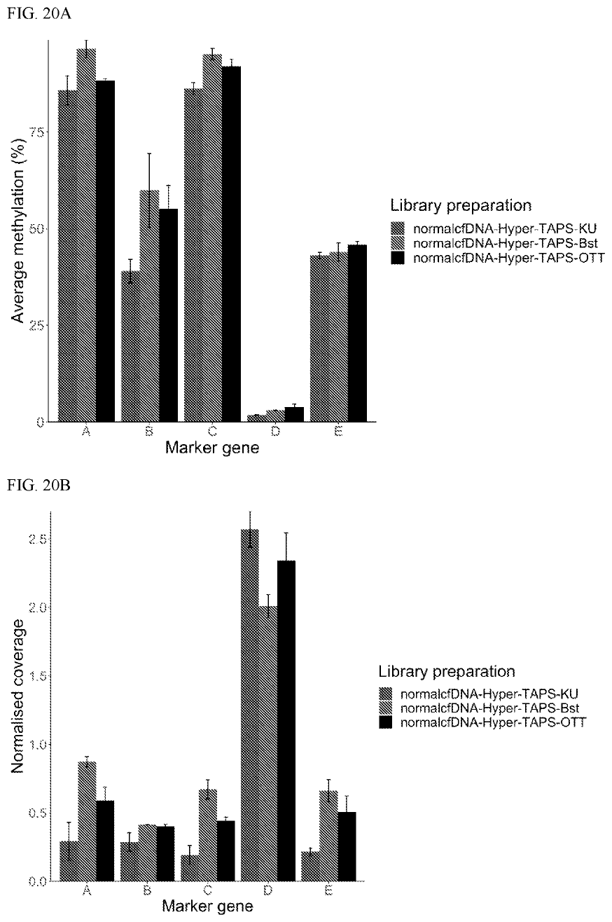

Furthermore, we show that the improved coverage with complementary strand synthesis step conditions using Bst or OneTaq and Tth (OTT) is also seen on selected highly methylated markers in a pool of normal cfDNA both in Whole Genome Sequencing (WGS) (FIG. 19A-B-improved coverage markers b, d, e, h, i) or hybridization capture targeted sequencing (FIG. 20A-B-improved coverage markers A-C, E).

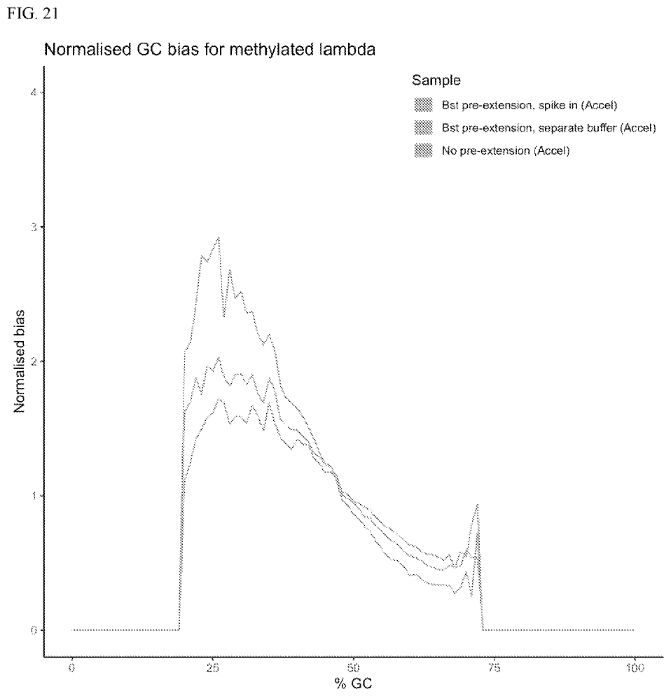

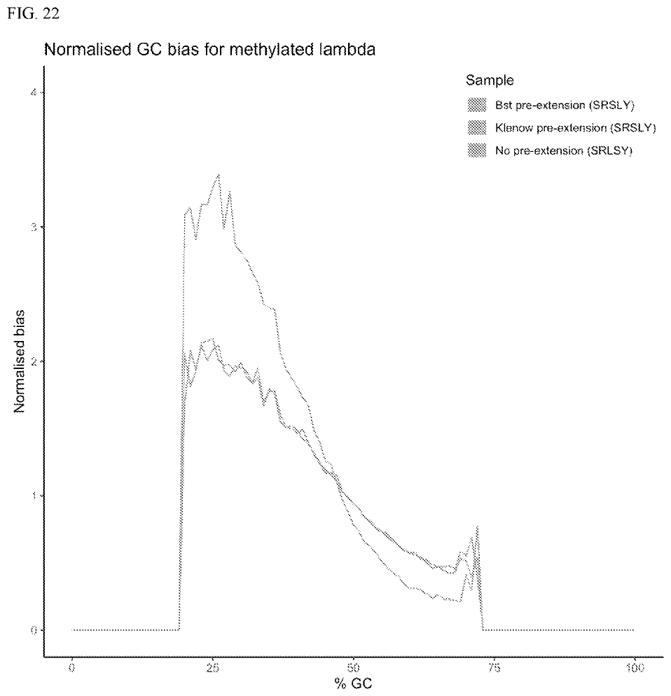

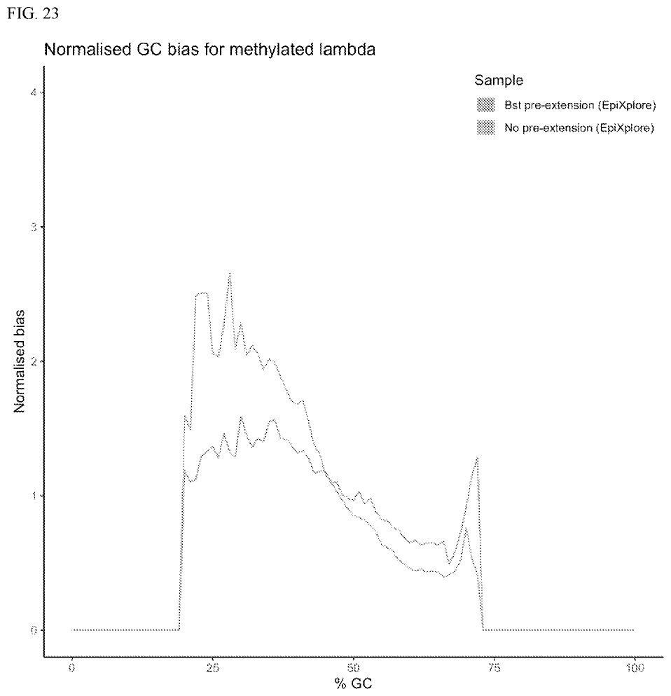

Selected complementary strand synthesis step options also showed improvement with Accel-NGS Methyl-Seq DNA library kit from Swift BioSciences, SRSLY NGS Library kit from Claret Bioscience, and EpiXplore™ Methylated DNA kit from Takara Bio as shown in FIGS. 21-23.

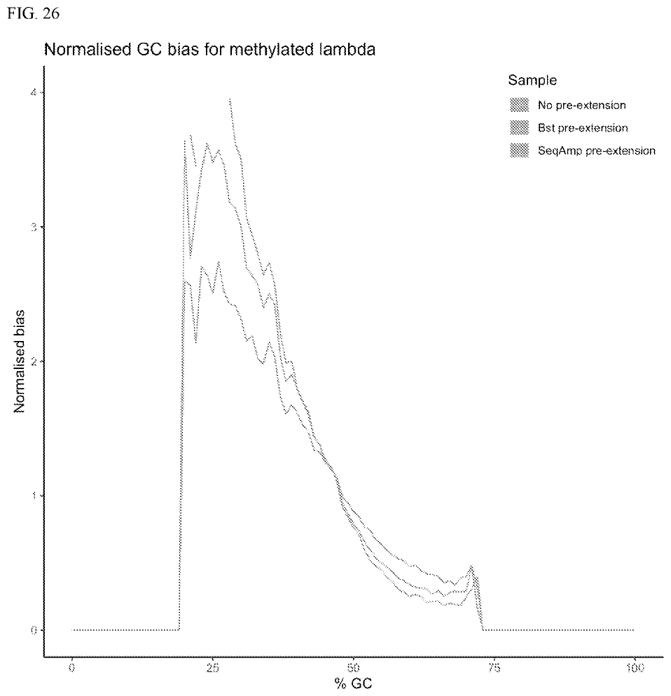

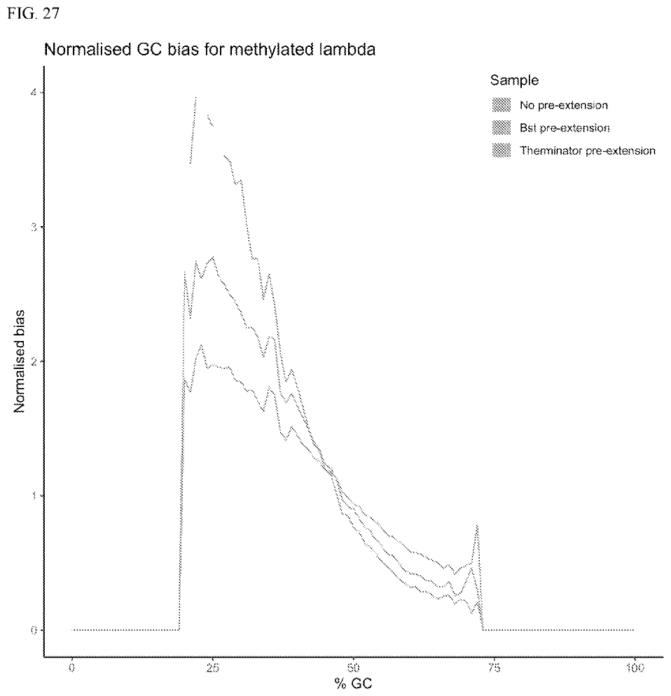

A number of other polymerases were screened which did not improve normalized GC bias when used for the complementary strand synthesis step under the test conditions described above. Examples of these polymerases include KAPA HiFi Uracil+, full-length Bst polymerase, Therminator polymerase, phi29 polymerase, AMV reverse transcriptase, Taq polymerase (NEB), NEB Q5U polymerase, NEB LongAmp Taq, Pyromark, Phusion U<SeqAmp, and ProtoScript™ reverse transcriptase. See, e.g., FIGS. 26 and 27 which provides results for the complementary strand synthesis step with SeqAmp and Therminator polymerases, respectively, as compared to Bst polymerase.

### Example 2

This example provides data related to optimization of conditions for conversion of 5-carboxylcytosine (5 caC) and/or 5-formylcytosine (5fC) residues in oxidized nucleic acid samples to dihydrouracil (DHU) residues via use of a borane reducing agent (for instance, pic-borane). These data demonstrate that the conversion of 5 caC to dihydrouracil (DHU) using borane reduction chemistry with previously established conditions or conditions described in Nature Biotechnology (37) 424-429 (2019) can be improved by including from 45% to 52.5% v/v of an organic solvent (e.g., dimethyl sulfoxide (DMSO)) in the reaction mixture. The data further show that in addition to using an increased amount of solvent in the reaction, improved results may be obtained using a reaction temperature of from 45 to 52.5 degrees C. and/or a shortened reaction time of from 45 to 60 minutes.

Compared to alternate borane reduction conditions, the use of a higher concentration of solvent (e.g., DMSO) in the reaction has multiple benefits. For instance, previous conditions that used 10% of DMSO resulted in significant bias in the amplification of the library after borane reaction. The use of reaction conditions with an increased solvent concentration, as described herein, can reduce this bias effectively. Alternate borane reduction condition optimizations have been attempted, including altering the conditions through increased temperatures, shorter reaction times, changes in borane concentrations, and use of different buffers and pH conditions. However, these previous optimization efforts have not resulted in the beneficial optimization of DMSO concentration demonstrated herein. For instance, alternate conditions with increased reaction temperature alone result in increased false positive rates and conditions which decreased the reaction time alone resulted in reduced conversion rates, as compared to conditions using increased solvent levels, which maintain low false positive rates and improved genomic coverage. Thus, the data show that solvent concentration mediates the effects of reducing reaction time (which generally reduces the conversion rate) and increasing the reaction temperature (which generally increases the false positive (FP) rate as well as bias).

Specifically, when reaction time alone was altered (shortened from 2 hours to 1 hour) without altering other conditions (37° C. with 10% DMSO), conversion rates dropped from 92% to 85% (measured using a spiked-in methylated pUC19). When reaction time was reduced from 2 hours to 1 hour along with an increase in reaction temperature to 50° C., the conversion rate (measured using a spiked-in methylated Lambda template) increased to approximately 94%, but this increase was accompanied by an increase in the false positive rate to approximately 2%. That represents an approximately 6 fold increase from the approximately 0.35% false positive rate observed in the standard conditions (10% DMSO, 37° C., 2 hours). Unexpectedly, when the concentration of DMSO was increased to 50% in the reactions conducted for 1 hour at 50° C., a high conversion rate was maintained (on average above 94%) while false positives were decreased on average to less than about 0.3%. The supporting data for these observations is provided in Table 4. GC Bias plots (not shown) were also generated for TAPS reactions conducted for 2 hours at 37° C. with 10% DMSO or 1 hour at 50° C. with 50% DMSO using different polymerases (Hifi HotStart KAPA Uracil plus, Bst 3.0 and OneTaq/Tth). The plots for the reactions using 50% DMSO were flatter than the plots for the reactions using 10% DMSO, demonstrating improved coverage and reduced GC bias for the reactions containing 50% DMSO. Additional data that further clarifies the optimal ranges for time, temperature and solvent concentration is discussed following Table 4.

TABLE 4
Results of varying time, temperature, and DMSO concentration

| | | | | Conversion | Conversion | |
|---|---|---|---|---|---|---|
| | Time | Temp. | DMSO | pUC19 | Lambda | FP |
| Sample | (Hours) | (° C.) | (V/V)% | Methylation | Methylation | Rate |
| S33 | 1 | 37 | 10 | 86.2 | | 0.28 |
| S34 | 1 | 37 | 10 | 85.8 | | 0.39 |
| S11 | 2 | 37 | 10 | 92.1 | | 0.36 |
| S12 | 2 | 37 | 10 | 92.7 | | 0.35 |
| S67 | 1 | 37 | 10 | | 81.0 | 0.10 |
| S68 | 1 | 37 | 10 | | 82.9 | 0.16 |
| S73 | 1 | 50 | 10 | | 94.2 | 1.93 |
| S74 | 1 | 50 | 10 | | 93.7 | 2.17 |
| S75 | 1 | 50 | 50 | | 94.5 | 0.26 |
| S76 | 1 | 50 | 50 | | 94.2 | 0.32 |

Unless otherwise noted, the experiments described herein compared optimized reaction conditions that varied DMSO concentrations, reaction temperatures, and reaction times to base reaction conditions. The base reaction conditions utilized a 50 μl reaction volume containing 50 ng of oxidized dsDNA, 100 mM buffer at pH 4.0 (5 μl), 100 mM Pic-borane in DMSO (5 μl) (providing 10% v/v DMSO), wherein the reaction was run for 2 hours at 37 degrees Celsius. A number of values for solvent concentration, time of reaction and temperature of reaction were evaluated to establish optimal ranges and are reported in the Tables below. A number of experimental parameters for the different conditions were examined and are reported in the Tables below.

Briefly, methylation conversion represents detection of C->T conversions following TAPS in fully methylated Lamba or partially methylated pUC19 spike-ins. The pUC19 DNA spike-in contains ˜20% methylation and is intended to represent real world conditions where less than 100% of the template is methylated. False positives are defined as the detection of C->T conversions on a fully un-methylated 2 kb spike-in. GC bias describes the dependence between fragment count (read coverage) and GC content found in Illumina sequencing data. GC dropout is a metric relating to the degree of sequencing bias in a sample, whereby samples with greater GC bias have a correspondingly higher GC dropout. For example, the Lambda GC dropout therefore represents the sequencing bias from the methylated Lambda DNA spike-in to the TAPS reaction where the majority of Cs are methylated.

The effect of increasing the concentration of DMSO in the reaction mixture was evaluated. TAPS reactions were run at 50° C. for 1 hour using varying DMSO concentrations, including 0%, 10%, 25%, 50%, 60%, and 75% DMSO v/v. The data are presented in Table 5. As can be seen, the TAPS reactions utilizing 50% DMSO had similar levels of conversion as 10% DMSO as assayed by Lambda methylation, improved levels of conversion as assayed by pUC19 methylation (which is more representative of real-world conditions), greatly improved false positive rates, and improved GC drop out metrics. The improvement with respect to GC bias was also observed in GC plots where the curve for the 50% DMSO reaction was flatter than the curve for the reactions containing lower amounts of DMSO. TAPS reaction containing 25% DMSO had good conversion rates, but relatively high false positive rates and higher GC bias as represented by GC drop out metrics. When the DMSO concentration was increased to 60% or 75%, the conversion rates declined.

TABLE 5
Effect of increased DMSO concentration in TAPS reaction mixtures.

| | | | PicB | KU | Conversion | Conversion | FP Rate | General | Lambda |
|---|---|---|---|---|---|---|---|---|---|
| | % | | Recovery | Yield | Lambda | pUC19 | (2 kb | GC | GC |
| Sample | DMSO | pH | (%) Final | (ng) | Methylation | methylation | Conversion) | Drop Out | Dropout |
| S15 | 0 | 4.07 | 13.2 | 119.8 | 96.8 | 6.5 | 2.01 | 4.6 | 25.2 |
| S16 | 0 | 4.07 | 12.8 | 104 | 97.1 | 5.8 | 2.02 | 5.1 | 26.1 |
| S17 | 10 | 4.28 | 13.1 | 73.2 | 97.2 | 5.8 | 1.83 | 5.3 | 26.8 |
| S18 | 10 | 4.28 | 12.8 | 76.2 | 95.2 | 6.4 | 1.88 | 4.9 | 24.2 |
| S19 | 25 | 4.55 | 12.5 | 49.0 | 97.4 | 6.2 | 1.12 | 4.5 | 23.0 |
| S20 | 25 | 4.55 | 16.4 | 88.2 | 97.5 | 6.3 | 1.24 | 4.6 | 23.0 |
| S21 | 50 | 5.51 | 13.9 | 76.6 | 97.1 | 9.5 | 0.26 | 3.3 | 14.1 |
| S22 | 50 | 5.51 | 12.6 | 141.2 | 97.3 | 11.1 | 0.26 | 3.0 | 12.2 |
| S23 | 60 | 6.05 | 12.9 | 138.2 | 93.6 | 10.3 | 0.14 | 3.2 | 12.7 |
| S24 | 60 | 6.05 | 10.4 | 116.2 | 72.9 | 8.3 | 0.10 | 3.5 | 13.2 |
| S25 | 75 | 6.79 | 10.8 | 101.4 | 34.0 | 4.7 | 0.13 | 2.8 | 9.3 |
| S26 | 75 | 6.79 | 11.7 | 131.0 | 21.4 | 3.4 | 0.08 | 1.9 | 6.8 |

The effect of increasing the temperature of reaction with 10% or 50% DMSO v/v was further evaluated. Conversion and false positive rates were measured for varying temperatures, including 20° C., 37° C., 45° C., and 50° C., with 10% and 50% DMSO v/v and a reaction time of 1 hour. This data is presented in Table 6. Conversion, false positive, and GC drop out metrics were also measured for higher temperatures, including 50° C., 75° C., and 100° C., each with 10% or 50% DMSO v/v. The data are presented in Table 7. Referring to Table 6, the data indicate that use of increased DMSO concentrations reduce the false positive rates. In particular, at reaction temperatures above 37° C., use of 50% DMSO in the reaction mixtures decreases the false positive rate as compared to the use of 10% DMSO. Referring to Table 7, the data indicate that the false positive rate begins to increase at reaction temperatures above 50° C. and there is also a decrease in recoverable DNA. Use of 50% DMSO also results in a reduction of GC bias at all temperatures evaluated as indicated by the GC dropout metrics. The effect on GC bias was confirmed by GC bias plots (not shown) which demonstrated flatter curves when 50% DMSO is included in the reaction mixtures.

TABLE 6
Effect of increased reaction temperature on TAPS reactions.

| | | | PicB | KU | Conversion_{—} | FP Rate |
|---|---|---|---|---|---|---|
| | Temp | % | Recovery | Yield | pUC19 | (2 kb |
| Sample | (° C.) | DMSO | (%) Final | (ng) | methylation | Conversion) |
| S05 | 50 | 10 | 11.97 | 91.2 | 93.2 | 2.27 |
| S06 | 50 | 10 | 15.75 | 101.6 | 94.1 | 2.36 |
| S07 | 50 | 50 | 15.75 | 236 | 94.2 | 0.33 |
| S08 | 50 | 50 | 15.14 | 226 | 94.2 | 0.35 |
| S13 | 37 | 10 | 47.08 | 218 | 84.1 | 0.23 |
| S14 | 37 | 10 | 47.96 | 214 | 86.3 | 0.27 |
| S15 | 37 | 50 | 19.4 | 292 | 74.0 | 0.11 |
| S16 | 37 | 50 | 20.5 | 280 | 73.5 | 0.12 |
| S17 | 20 | 10 | 55.44 | 250 | 17.5 | 0.09 |
| S18 | 20 | 10 | 78.32 | 370 | 25.1 | 0.09 |
| S19 | 20 | 50 | 25.74 | 316 | 7.9 | 0.08 |
| S20 | 20 | 50 | 22.97 | 370 | 8.9 | 0.09 |
| S21 | 45 | 10 | 16.19 | 188.6 | 94.0 | 1.19 |
| S22 | 45 | 10 | 16.9 | 188.8 | 93.1 | 1.28 |
| S23 | 45 | 50 | 17.78 | 286 | 92 | 0.26 |
| S24 | 45 | 50 | 16.63 | 324 | 92.5 | 0.25 |

TABLE 7
Effect of high reaction temperatures on TAPS reactions.

| | | | PicB | KU | Conversion_{—} | Conversion_{—} | FP Rate | General | Lambda |
|---|---|---|---|---|---|---|---|---|---|
| | Temp | % | Recovery | Yield | Lambda | pUC19 | (2 kb | GC | GC |
| Sample | (° C.) | DMSO | (%) Final | (ng) | Methylation | methylation | Conversion) | Drop Out | Dropout |
| S05 | 50 | 10 | 13.99 | 113.2 | 96.9 | 6.2 | 2.7 | 4.6 | 26.8 |
| S06 | 50 | 10 | 13.64 | 127 | 97.2 | 6.6 | 2.9 | 4.5 | 25.5 |
| S07 | 50 | 50 | 13.82 | 310 | 97.4 | 10.6 | 0.3 | 2.2 | 11.3 |
| S08 | 50 | 50 | 12.32 | 482 | 97.2 | 9.9 | 0.3 | 2.3 | 12.1 |
| S13 | 75 | 10 | 10.03 | 12.66 | 68.8 | 8.3 | 8.7 | 8.3 | 39.7 |
| S14 | 75 | 10 | 7.3 | 11.66 | 79.6 | 8.3 | 7.8 | 8.2 | 38.6 |
| S15 | 75 | 50 | 9.06 | 22.2 | 96.9 | 6.2 | 1.1 | 4.6 | 22.1 |
| S16 | 75 | 50 | 4.14 | 10.64 | 91.2 | 8.1 | 1.5 | 3.9 | 16.2 |
| S21 | 100 | 10 | 1.09 | 2.3 | 15.4 | 1.9 | 0 | 5.4 | 22.0 |
| S22 | 100 | 10 | 0.65 | 1.55 | 16.8 | 0.5 | 0 | 4.5 | 21.0 |
| S23 | 100 | 50 | 0.78 | 2.36 | 2.6 | 0.7 | 0.2 | 0.9 | 5.7 |
| S24 | 100 | 50 | 0.66 | 1.896 | 23.4 | 4.4 | 1.1 | 0.1 | 5.5 |

The effect of time of reaction with 10% or 50% DMSO v/v was evaluated. Conversion and false positive rates were measured for reactions run for 15 min or 1 hour, at 37° C. or 50° C., each with 10% or 50% DMSO v/v. Conversion and GC bias (represented as GC dropout metrics) were also measured for reactions run for 1 hour, 2 hours, 6 hours, or 24 hours, each with 50% DMSO v/v and at a reaction temperature of 50° C. The data are presented in Tables 8 and 9. Referring to Table 8, the data indicate that there is no benefit of increased DMSO for shorter reaction times as evidenced by the lower conversion rates for the reactions conducted for 15 minutes at either 37° C. or 50° C. and with either 10% or 50% DMSO v/v. Referring to Table 9, the data indicate that the longer reaction times of 2 hours, 6 hours and 24 hours begin to increase the false positive rate and decrease the yield. The GC dropout metrics also indicate that longer reaction times are generally associated with increased GC bias. The effect on GC bias was confirmed by GC bias plots (not shown) which demonstrated flatter curves, especially for the 1 and 2 hour reaction as compared to the 6 and 24 hour reactions.

TABLE 8
Effect of time on TAPS reactions at different DMSO concentrations.

| | | | | | PicB | KU | Conversion_{—} | FP Rate |
|---|---|---|---|---|---|---|---|---|
| | Temp | Rxn | | % | Recovery | Yield | pUC19 | (2 kb |
| Sample | (° C.) | Time | | DMSO | (%) Final | (ng) | methylation | Conversion) |
| S01 | 50 | 15 | m | 10 | 15.31 | 214 | 85.6 | 0.50 |
| S02 | 50 | 15 | m | 10 | 14.39 | 177.4 | 85.5 | 0.50 |
| S03 | 50 | 15 | m | 50 | 17.38 | 292 | 78.1 | 0.11 |
| S04 | 50 | 15 | m | 50 | 17.16 | 274 | 79.3 | 0.17 |
| S05 | 50 | 1 | h | 10 | 11.97 | 91.2 | 93.2 | 2.27 |
| S06 | 50 | 1 | h | 10 | 15.75 | 101.6 | 94.1 | 2.36 |
| S07 | 50 | 1 | h | 50 | 15.75 | 236 | 94.2 | 0.33 |
| S08 | 50 | 1 | h | 50 | 15.14 | 226 | 94.2 | 0.35 |
| S09 | 37 | 15 | m | 10 | 48.84 | 274 | 33.8 | 0.10 |
| S10 | 37 | 15 | m | 10 | 42.46 | 274 | 30.5 | 0.14 |
| S11 | 37 | 15 | m | 50 | 20.42 | 334 | 27.9 | 0.09 |
| S12 | 37 | 15 | m | 50 | 18.88 | 264 | 29.0 | 0.09 |
| S13 | 37 | 1 | h | 10 | 47.08 | 218 | 84.1 | 0.23 |
| S14 | 37 | 1 | h | 10 | 47.96 | 214 | 86.3 | 0.27 |
| S15 | 37 | 1 | h | 50 | 19.4 | 292 | 74.0 | 0.11 |
| S16 | 37 | 1 | h | 50 | 20.5 | 280 | 73.5 | 0.12 |

TABLE 9
Effect of longer times on TAPS reactions.

| | | PicB | KU | Conversion_{—} | Conversion_{—} | FP Rate | General | Lambda |
|---|---|---|---|---|---|---|---|---|
| | Rxn | Recovery | Yield | Lambda | pUC19 | (2 kb | GC | GC |
| Sample | Time | (%) Final | (ng) | Methylation | methylation | Conversion) | Drop Out | Dropout |
| S05 | 1 h | 17.38 | 260 | 97.1 | 9.7 | 0.23 | 3.31 | 14.07 |
| S06 | 1 h | 16.06 | 332 | 97.0 | 8.3 | 0.50 | 3.44 | 15.38 |
| S11 | 2 h | 16.41 | 344 | 97.3 | 9.3 | 0.39 | 3.29 | 14.00 |
| S12 | 2 h | 15.93 | 230 | 97.4 | 9.7 | 0.46 | 3.22 | 13.78 |
| S17 | 6 h | 12.1 | 330 | 97.8 | 9.6 | 1.15 | 3.79 | 15.65 |
| S18 | 6 h | 15.36 | 422 | 97.7 | 10.2 | 1.11 | 3.96 | 15.07 |
| S23 | 24 h | 15.31 | 73.6 | 97.6 | 9.2 | 1.96 | 4.72 | 19.50 |
| S24 | 24 h | 13.2 | 84.6 | 97.5 | 9.1 | 2.01 | 4.56 | 18.37 |

Additional experiments were conducted to further define the optimum ranges for DMSO concentration (measuring 35%, 40%, 45%, 47.5%, 50%, 52.5%, 55%, and 60% DMSO v/v), temperature of reaction (measuring 45° C., 47.5° C., 50° C., 52.5° C., and 55° C.), and time of reaction (measuring 45 min, 50 min, 55 min, 1 hour, and 2 hours) The collated data are presented in Table 10. From these data, it can be determined that the optimal range for DMSO is from 45% to 52.5% v/v. Outside of this range, either a decrease in conversion or increase in false positive rate is observed. It can further be determined that the optimal time of reaction is from 45 minutes to 1 hour. Outside of this range, an increase in false positive rate is observed while the conversion remains high. Finally, it can be determined that optimal reaction temperature is from 45° C. to 52.5° C. Outside of this range, an increase in false positive rate is observed.

TABLE 10
Collated data for effects of DMSO concentration, time and temperature on TAPS reactions.

| | | | | | PicB | KU | Conversion | Conversion | FP Rate | General | Lambda |
|---|---|---|---|---|---|---|---|---|---|---|---|
| | Temp | Rxn | | % | Recovery | Yield | Lambda | pUC19 | (2 kb | GC | GC |
| Sample | (° C.) | Time | | DMSO | (%) Final | (ng) | Methylation | Methylation | Conversion) | Dropout | Dropout |
| SW01 | 50 | 1 | h | 50 | 22.26 | 356 | 97.5 | 10.5 | 0.71 | 2.17 | 2.17 |
| SW02 | 50 | 1 | h | 50 | 23.23 | 272 | 97.4 | 10.8 | 0.25 | 2.26 | 2.26 |
| SW03 | 50 | 1 | h | 35 | 24.01 | 268 | 97.5 | 10.0 | 0.71 | 2.41 | 2.41 |
| SW04 | 50 | 1 | h | 35 | 23.69 | 173 | 97.3 | 8.8 | 0.64 | 2.54 | 2.54 |
| SW05 | 50 | 1 | h | 40 | 19.37 | 240 | 97.4 | 9.3 | 0.51 | 2.16 | 2.16 |
| SW06 | 50 | 1 | h | 40 | 19.23 | 286 | 97.4 | 10.0 | 0.46 | 2.43 | 2.43 |
| SW07 | 50 | 1 | h | 45 | 16.19 | 382 | 97.4 | 10.2 | 0.43 | 2.34 | 2.34 |
| SW08 | 50 | 1 | h | 45 | 15.92 | 494 | 97.2 | 11.2 | 0.30 | 2.18 | 2.18 |
| SW09 | 50 | 1 | h | 47.5 | 19.09 | 386 | 97.4 | 11.6 | 0.31 | 1.79 | 1.79 |
| SW10 | 50 | 1 | h | 47.5 | 17.99 | 334 | 97.5 | 10.9 | 0.28 | 1.90 | 1.90 |
| SW11 | 50 | 1 | h | 52.5 | 16.97 | 378 | 96.5 | 11.0 | 0.23 | 2.10 | 2.10 |
| SW12 | 50 | 1 | h | 52.5 | 19.32 | 292 | 96.5 | 11.5 | 0.22 | 2.00 | 2.00 |
| SW13 | 50 | 1 | h | 55 | 17.66 | 314 | 94.2 | 11.4 | 0.22 | 1.97 | 1.97 |
| SW14 | 50 | 1 | h | 55 | 14.72 | 388 | 94.1 | 11.2 | 0.20 | 2.11 | 2.11 |
| SW15 | 50 | 1 | h | 60 | 10.86 | 352 | 77.0 | 9.1 | 0.16 | 2.65 | 2.65 |
| SW16 | 50 | 1 | h | 60 | 14.26 | 468 | 76.7 | 9.3 | 0.14 | 2.43 | 2.43 |
| SW17 | 45 | 1 | h | 50 | 19.83 | 396 | 96.6 | 12.0 | 0.16 | 1.81 | 1.81 |
| SW18 | 45 | 1 | h | 50 | 19.92 | 464 | 96.6 | 11.5 | 0.16 | 1.78 | 1.78 |
| SW19 | 47.5 | 1 | h | 50 | 20.01 | 135.2 | 97.2 | 11.5 | 0.20 | 1.90 | 1.90 |
| SW20 | 47.5 | 1 | h | 50 | 19.64 | 352 | 96.9 | 12.4 | 0.66 | 1.94 | 1.94 |
| SW21 | 52.5 | 1 | h | 50 | 17.2 | 406 | 97.6 | 11.1 | 0.29 | 1.92 | 1.92 |
| SW22 | 52.5 | 1 | h | 50 | 15.18 | 392 | 97.7 | 11.8 | 0.29 | 2.16 | 2.16 |
| SW23 | 55 | 1 | h | 50 | 18.03 | 290 | 97.7 | 11.2 | 0.35 | 2.10 | 2.10 |
| SW24 | 55 | 1 | h | 50 | 14.08 | 386 | 97.7 | 10.0 | 0.46 | 2.40 | 2.40 |
| SW25 | 50 | 45 | min | 50 | 21.3 | 384 | 97.1 | 10.9 | 0.24 | 2.01 | 2.01 |
| SW26 | 50 | 45 | min | 50 | 15 | 242 | 97.1 | 10.8 | 0.20 | 2.08 | 2.08 |
| SW27 | 50 | 50 | min | 50 | 13.43 | 254 | 97.0 | 10.3 | 0.22 | 2.03 | 2.03 |
| SW28 | 50 | 50 | min | 50 | 17.62 | 292 | 97.0 | 11.6 | 0.25 | 2.32 | 2.32 |
| SW29 | 50 | 55 | min | 50 | 14.81 | 162.8 | 97.3 | 10.8 | 0.22 | 2.16 | 2.16 |
| SW30 | 50 | 55 | min | 50 | 12.65 | 268 | 97.3 | 10.6 | 0.21 | 2.08 | 2.08 |
| SW31 | 50 | 120 | min | 50 | 13.25 | 278 | 97.8 | 11.1 | 0.49 | 2.16 | 2.16 |
| SW32 | 50 | 120 | min | 50 | 19.04 | 430 | 97.4 | 13.1 | 0.41 | 2.75 | 2.75 |

Finally, the optimized conditions of 50% v/v DMSO and 1 hour reaction time at 50 degrees Celsius (designated ESI-NEW in Table 11) were compared to several sets of conditions, some of which have been previously utilized (designated in Table 10 as ESI-OLD (10% DMSO, 37° C., 1 hour), CS (8% DMSO, 37° C., 1 or 4 hours), and NB (Nature Biotechnology (37) 424-429 (2019) (Pyridine Borane (PyB) or Pic-Borane (PicB), 0 or 50% DMSO, 37° C. or 50° C., 1, 3 or 16 hours). The data are presented in Table 11. These data show that the optimized conditions (ESI-NEW) provide the highest conversion levels of any of the conditions tested especially for the pUC19 template (i.e., above 96% for Lambda methylation and above 9.5% for pUC19 methylation). The optimized conditions also provide the highest yields, while maintaining low false positive rates and good GC drop out metrics.

TABLE 11
Comparison of different TAPS reaction conditions.

| | | | | Pic | Buffer | PicB | KU | Conversion | Conversion | FP Rate | General | Lambda |
|---|---|---|---|---|---|---|---|---|---|---|---|---|
| | Temp | Rxn | % | Conc | Conc | Recovery | Yield | Lambda | pUC19 | (2 kb | GC | GC |
| Sample | (° C.) | Time | DMSO | (mM) | (mM) | (%) Final | (ng) | Methylation | methylation | Conversion) | Drop Out | Dropout |
| S19-SW-ESI-OLD | 37 | 1 h | 10 | 100 | 100 | 57.64 | 312 | 93.1 | 3.6 | 0.11 | 1.42 | 22.11 |
| S20-SW-ESI-OLD | 37 | 1 h | 10 | 100 | 100 | 61.16 | 352 | 93.0 | 4.0 | 0.08 | 1.34 | 20.82 |
| S21-SW-CS | 37 | 1 h | 8 | 100 | 100 | 77 | 149.6 | 93.0 | 4.1 | 0.12 | 2.71 | 21.6 |
| S22-SW-CS | 37 | 1 h | 8 | 100 | 100 | 72.16 | 230 | 94.3 | 3.7 | 0.14 | 2.72 | 22.46 |
| S23-SW-NB-PyB | 37 | 1 h | 0 | 1000 | 600 | 80.08 | 214 | 33.0 | 2.4 | 0.09 | 0.81 | 15.95 |
| S24-SW-NB-PyB | 37 | 1 h | 0 | 1000 | 600 | 82.72 | 284 | 33.4 | 2.7 | 0.07 | 2.67 | 15.9 |
| S25-SW-NB-PyB | 37 | 1 h | 0 | 1000 | 100 | 65.12 | 258 | 46.0 | 4.6 | 0.06 | 2.12 | 13.5 |
| S26-SW-NB-PyB | 37 | 1 h | 0 | 1000 | 100 | 53.68 | 286 | 46.0 | 5.0 | 0.05 | 1.34 | 13.21 |
| S27-SW-CS | 37 | 4 h | 8 | 100 | 100 | 63.36 | 310 | 95.2 | 4.3 | 0.62 | 2.15 | 22.37 |
| S28-SW-CS | 37 | 4 h | 8 | 100 | 100 | 69.08 | 296 | 95.7 | 3.8 | 0.41 | 2.8 | 23.15 |
| S29-SW-NB-PyB | 37 | 16 h | 0 | 1000 | 600 | 67.32 | 198.4 | 95.5 | 4.8 | 0.3 | 4.49 | 27.12 |
| S30-SW-NB-PyB | 37 | 16 h | 0 | 1000 | 600 | 75.24 | 356 | 95.5 | 4.9 | 0.29 | 4.49 | 27.58 |
| S31-SW-ESI-New | 50 | 1 h | 50 | 100 | 100 | 23.32 | 486 | 96.2 | 9.7 | 0.38 | 2.92 | 12.87 |
| S32-SW-ESI-New | 50 | 1 h | 50 | 100 | 100 | 21.52 | 400 | 96.3 | 9.6 | 0.28 | 2.79 | 12.8 |
| S33-SW-NB-PyB | 50 | 1 h | 0 | 1000 | 100 | 47.52 | 78.6 | 86.0 | 4.1 | 0.28 | 5.77 | 26.58 |
| S34-SW-NB-PyB | 50 | 1 h | 0 | 1000 | 100 | 52.36 | 62.4 | 87.0 | 4.3 | 0.2 | 7.11 | 27.31 |
| S35-SW-NB- | 50 | 1 h | 50 | 1000 | 100 | 13.73 | 113.8 | 94.3 | 6.4 | 1.54 | 3.95 | 19.58 |
| 50DMSO-PyB | | | | | | | | | | | | |
| S36-SW-NB- | 50 | 1 h | 50 | 1000 | 100 | 16.9 | 134 | 94.8 | 6.8 | 1.55 | 4.54 | 19.58 |
| 50DMSO-PyB | | | | | | | | | | | | |
| S37-SW-NB- | 50 | 3 h | 50 | 1500 | 300 | 15.18 | 144 | 31.6 | 5.5 | 0.32 | 0.22 | 1.26 |
| 50DMSO-PicB | | | | | | | | | | | | |
| S38-SW-NB- | 50 | 3 h | 50 | 1500 | 300 | 12.19 | 195.2 | 21.0 | 4.3 | 0.25 | 0.07 | 0.07 |
| 50DMSO-PicB | | | | | | | | | | | | |

What is claimed is:

1. A method for amplifying a target nucleic acid molecule comprising dihydrouracil (DHU) residues comprising:
synthesizing one or more complementary strands of the target nucleic acid comprising DHU residues with a first polymerase or polymerase mixture that is tolerant of DHU residues and/or products resulting from the introduction of the DHU residues and/or the TAPS process to provide a target nucleic acid mixture comprising the target nucleic acid comprising DHU residues and one or more complementary strands; and
exponentially amplifying the target nucleic acid mixture to provide amplified target nucleic acid.

2. The method of claim 1, wherein the first polymerase or polymerase mixture has an error rate of greater than 5.0×10⁻⁵.

3. The method of any one of claims 1 to 2, wherein the first polymerase or polymerase mixture is selected from the group consisting of Bst3.0 polymerase, Sulpholobus polymerase IV, a combination of Bst3.0 polymerase and Sulpholobus polymerase IV, Klenow polymerase, Klenow exo-polymerase, Polk polymerase, Mu-mLV reverse transcriptase, SD polymerase, Tth polymerase, OneTaq polymerase, a combination of OneTaq and Tth polymerase, 5D4 polymerase, a 5D4 polymerase blend with Taq polymerase, and SD polymerase.

4. The method of any one of claims 1 to 3, wherein the first polymerase is thermolabile.

5. The method of any one of claims 1 to 3, wherein the first polymerase is thermostable.

6. The method of any one of claims 1 to 3, wherein the step of exponentially amplifying the complementary strand of the target nucleic acid utilizes the first polymerase or polymerase mixture that is tolerant of DHU residues and/or products resulting from the introduction of the DHU residues and/or the TAPS process.

7. The method of any one of claims 1 to 5, wherein the step of exponentially amplifying the pre-amplified target nucleic acid utilizes a second polymerase or polymerase mixture that is different from the first polymerase or polymerase mixture.

8. The method of claim 7, wherein the second polymerase or polymerase mixture has an error rate of less than 5.0×10⁻⁵.

9. The method of claim 7, wherein the second polymerase or polymerase mixture has an error rate of less than 1.0×10⁻⁶.

10. The method of any one of claims 7 to 9, wherein the second polymerase is selected from the group consisting of GoTaq polymerase and KAPA HiFi Uracil+ polymerase.

11. The method of any one of claims 7 to 9, wherein the polymerase having an error rate of less than 5.0×10⁻⁵ is thermostable.

12. The method of any one of claims 7 to 10, wherein the first polymerase and the second polymerase are provided in a mastermix.

13. The method of any one of claims 1 to 12, wherein synthesizing a complementary strand of the target nucleic acid comprising DHU residues with a first polymerase or polymerase mixture further comprises synthesis in a buffer comprising from about 0.5-0.75 mM MnSO₄.

14. The method of any one of claims 1 to 13, further comprising quantifying the amplified target nucleic acid.

15. The method of any one of claims 1 to 14, further comprising the step of sequencing the exponentially amplified target nucleic acid.

16. The method of any one of claim 15, wherein the target nucleic acid comprising DHU residues has sequencing library adapters ligated to each end.

17. The method of claim 16, wherein the sequencing library adapters comprise an index sequence.

18. The method of any one of claims 16 to 17, wherein the sequencing library adapters comprise sequences complementary to sequencing primers.

19. The method of any one of claims 15 to 18, wherein the sequencing library adapters comprise sequences complementary to indexing primers.

20. The method of any one of claims 15 to 19, wherein the step of synthesizing a complementary strand of the target nucleic acid comprising DHU residues further comprises annealing forward and/or reverse primer(s) to the sequencing library adapters.

21. The method of any one of claims 15 to 20, wherein the step of exponentially amplifying the complementary strand of the target nucleic acid comprises annealing library amplification primers to the pre-amplified target nucleic acid.

22. The method of any one of claims 15 to 21, wherein the sequencing is performed by massively parallel sequencing.

23. The method of any one of claims 1 to 22, wherein the target nucleic acid molecule comprising DHU is produced by a process comprising contacting an oxidized nucleic acid sample comprising 5-carboxylcytosine (5 caC) and/or 5-formylcytosine (5fC) with a borane reducing agent.

24. The method of claim 23, wherein the borane reducing agent comprises an agent selected from the group consisting of 2-picoline borane (pic-BH3), borane, sodium borohydride, sodium cyanoborohydride, and sodium triacetoxyborohydride.

25. The method of any one of claims 23 to 24, wherein the step of contacting the oxidized nucleic acid sample comprising 5 caC and/or 5fC with a borane reducing agent further comprises reacting the oxidized nucleic acid sample with the borane reducing agent in a reaction mixture comprising from 45.0% to 52.5% DMSO by volume.

26. The method of any one of claims 23 to 25, wherein the step of contacting the oxidized nucleic acid sample comprising 5 caC and/or 5fC with a borane reducing agent further comprises reacting the oxidized nucleic acid sample with the borane reducing agent at a temperature of from 45.0 degrees Celsius to 52.5 degrees Celsius.

27. The method of any one of claims 23 to 26, wherein the step of contacting the oxidized nucleic acid sample comprising 5 caC and/or 5fC with a borane reducing agent further comprises reacting the oxidized nucleic acid sample with the borane reducing agent for a period of time of from 45 to 60 minutes.

28. A method for converting 5-carboxylcytosine (5 caC) and/or 5-formylcytosine (5fC) to dihydrouracil (DHU) comprising contacting a nucleic acid sample comprising 5 caC and/or 5fC with a borane reducing agent in a reaction mixture comprising from 45.0% to 52.5% DMSO by volume.

29. The method of claim 28, further comprising reacting the oxidized nucleic acid sample with the borane reducing agent at a temperature of from 45.0 degrees Celsius to 52.5 degrees Celsius.

30. The method of any one of claims 28 to 29, further comprising reacting the oxidized nucleic acid sample with the borane reducing agent for a period of time from 45 to 60 minutes.

31. The method of any one of claims 28 to 30, wherein the borane reducing agent comprises an agent selected from the group consisting of 2-picoline borane (pic-BH3), borane, sodium borohydride, sodium cyanoborohydride, and sodium triacetoxyborohydride.

32. The method of claim 31, wherein the borane reducing agent comprises sodium borohydride.

33. The method of claim 31, wherein the borane reducing agent comprises sodium cyanoborohydride.

34. The method of claim 31, wherein the borane reducing agent comprises sodium triacetoxyborohydride.

35. The method of claim 31, wherein the borane reducing agent comprises 2-picoline borane.

36. The method of any one of claims 28 to 35, comprising contacting the nucleic acid sample with an oxidizing agent prior to contacting with a borane reducing agent.

37. The method of claim 36, wherein the oxidizing agent is a ten-eleven translocation (TET) enzyme.

38. The method of claim 37, wherein the TET enzyme comprises human TET1, human TET2, human TET3, murine TET1, murine TET2, murine TET3, Naegleria TET (NgTET), Coprinopsis cinerea (CcTET), or derivatives or analogues thereof.

39. The method of claim 36, wherein the oxidizing agent comprises a chemical oxidizing agent.

40. The method of claim 39, wherein the chemical oxidizing agent comprises manganese oxide (MnO₂), potassium ruthenate (K₂RuO₄), potassium perruthenate (KRuO₄) or Cu(II)/TEMPO.

41. The method of any one of claims 36 to 40, further comprising adding a blocking group to one or more modified cytosines in the nucleic acid sample.

42. The method of any one of claims 28 to 41, further comprising sequencing the nucleic acid sample after contacting with the borane reducing agent to identify converted cytosine bases.
